(19) United States
(12) Patent Application Publication
(10) Pub. No.: US 2016/0030603 A1
(43) Pub. Date: Feb. 4, 2016

(54) DETECTION OF ACUTE RENAL ALLOGRAFT REJECTION

(71) Applicant: WESTFAELISCHE WILHELMS-UNIVERSITAET MUENSTER, Muenster (DE)

(72) Inventors: Stefan Reuter, Muenster (DE); Klaus Tiemann, Feldafing (DE)

(21) Appl. No.: 14/776,720

(22) PCT Filed: Mar. 13, 2014
(86) PCT No.: PCT/EP2014/054992

§ 371 Date: Sep. 14, 2015

(30) Foreign Application Priority Data
Mar. 15, 2013 (EP) 13159604.1

(51) Int. Cl. A61K 49/22

(52) U.S. Cl. CPC A61K 49/221; A61K 49/223

(57) ABSTRACT

The invention provides a method of detecting acute renal allograft rejection in a subject as well as a method of stratifying a subject received a renal allograft and undergoing immunosuppressive therapy for alteration of the therapy. These methods comprise administering to the subject a probe capable of specifically binding to T lymphocytes, the probe being detectable by ultrasound. An allotransplanted kidney of the subject is exposed to ultrasound. The level of T lymphocytes in the kidney is detected. An elevated level of T lymphocytes in the kidney indicates an increased risk of renal allograft rejection and/or that the subject is in need of an alteration of the immunosuppressive therapy.

### FIELD OF THE INVENTION

The present invention relates to the detection of acute renal allograft rejection, and in particular the early detection of such rejection, in a subject. Provided is also a method of treating renal allograft rejection.

### BACKGROUND OF THE INVENTION

The following discussion of the background of the invention is merely provided to aid the reader in understanding the invention and is not admitted to describe or constitute prior art to the present invention.

With regard to quality of life, morbidity and mortality, renal transplantation is currently the best available method of treating patients suffering from end-stage renal failure. At present, renal grafts are the most common solid organ transplants world-wide. Renal transplantation is an attractive method of treatment, which is, however, limited by the risk of organ rejection. During the first months following transplantation, organ transplants are mainly at risk of acute allograft rejection, as well as the consequences of ischaemia/reperfusion injury. To prevent these events, immunosuppression is effected.

Post-transplantationally acute allograft rejections stand out by for instance primary malfunction of the transplant. Later on more than 50% of the rejection episodes occur at subclinical levels, without acute impaired renal function, and are thus only detected at an advanced stage—for example via a non-specific increase in creatinine levels. The gold standard in diagnosis of allograft rejection has so far been renal biopsy. Being an invasive procedure, biopsy is irksome to the patient and in addition involves the risk of significant graft injury or even loss. Moreover, the limited sampling sites (randomly chosen, and exceedingly small portions of tissue) may lead to false negative results, such as when rejection is focal or patchy.

The frequency of episodes of acute allograft rejection as well as the severity of episodes are unambiguous negative prognostic factors with regard to long-term graft survival. Only early detection of allograft rejection allows a swift escalation of immunosuppressive therapy, or a switch to a more effective therapeutic regimen, as in the case of steroid-refractory rejection, in order to efficiently minimize transplant damage.

Reuter et al. (The Journal of Nuclear Medicine [2010] 51, 10, 1644-1652; PLoS ONE [2009] 4, 4, e5296) applied ¹⁸F-Fluorodesoxyglucose, and detected its enrichment in inflamed areas using positron emission tomography. Hitchens et al. (Magn. Reson. Med [2011] 65, 4, 1144-1153) have detected macrophage accumulation in a kidney rejection model by means of magnetic resonance imaging based on the application of a ¹⁹F labelled crown ether that labelled macrophages in vivo.

In particular in view of the background of limited availability of organs, it would be advantageous to be able to detect and treat acute allograft rejection with high sensitivity and specificity as early as possible.

### SUMMARY OF THE INVENTION

The present disclosure can be taken to generally relate to the detection of acute renal allograft rejection in a subject that/who has received a renal transplant, and in particular to early detection of acute renal allograft rejection. In one aspect, provided is the identification of one or more subjects that/who are at higher risk for developing renal allograft rejection. A respective subject is typically at higher risk of acute renal allograft rejection than a comparable healthy subject, e.g. a subject of comparable age and/or gender, and possibly of comparable medical history. More specifically, the present invention provides inter alia a method for assessing the likelihood that a subject will develop a condition associated with renal allograft rejection and a method of stratification for risk of occurrence of acute renal allograft rejection. In some embodiments such a method is a method of risk stratification with regard to renal allograft rejection. In one aspect, provided is the diagnosis of occurrence of renal allograft rejection in one or more subjects, including diagnosis of occurrence of acute renal allograft rejection. Any such method may allow deciding on an appropriate therapeutic regimen for a subject. Provided is also a method of treating a subject, such as a patient, suffering from acute renal allograft rejection. A method according to the invention may include staging, monitoring, categorizing and/or determination of a subject's risk of acute renal allograft rejection, as well as staging, monitoring, categorizing and/or determination of further diagnosis and treatment regimens in a subject that is at an early stage of acute renal allograft rejection.

The methods and uses provided in this document are based on employing ultrasound at the region of the transplanted kidney after the subject has been administered a probe that is both capable of specifically binding to T lymphocytes and capable of being detected by ultrasound. In the context of the methods disclosed herein binding to T lymphocytes may be achieved using any desired technique. In some embodiments the probe may have a molecule or moiety coupled thereto, which has an affinity for T lymphocytes that renders it capable of specifically binding to T lymphocytes.

According to a first aspect, the present invention provides a method of detecting acute renal allograft rejection in a subject. The method includes administering a probe to the subject. The probe is capable of specifically binding to T lymphocytes. The probe is furthermore detectable by applying ultrasound. The method also includes exposing an allotransplanted kidney of the subject to ultrasound. Further the method includes detecting the level of T lymphocytes in the kidney. Generally the level of T lymphocytes in the kidney is being detected by detecting signals generated by exposing the kidney to ultrasound. These signals are generally echoes formed by reflection of the ultrasound in structures of the allotransplanted kidney. Typically a number of these echoes are induced and/or enhanced by the probe. An elevated level of T lymphocytes in the kidney indicates acute renal allograft rejection.

In some embodiments the method according to the first aspect essentially consists of administering the probe to the subject, exposing an allotransplanted kidney of the subject to ultrasound, and detecting the level of T lymphocytes in the kidney. In one embodiment the method according to the first aspect consists only of administering the probe to the subject, exposing an allotransplanted kidney of the subject to ultrasound and detecting the level of T lymphocytes in the kidney. In any such embodiment an elevated level of T lymphocytes in the kidney indicates acute renal allograft rejection.

In some embodiments of the method according to the first aspect detecting the level of T lymphocytes in the kidney is carried out by means of an imaging technique. In some embodiments detecting the level of T lymphocytes is performed via a real-time measurement. According to a particular embodiment of the method according to the first aspect exposing the allotransplanted kidney to ultrasound is carried out using a high intensity focused ultrasound technique.

In some embodiments of the method according to the first aspect the probe has a binding molecule immobilized thereon. The binding molecule is specific for T lymphocytes. In some embodiments of the method according to the first aspect the binding molecule is specific for a molecule or for a moiety present on T lymphocytes such as CD3. The binding molecule specific for a molecule or for a moiety present on T lymphocytes may also be specific for CD4. In some embodiments the binding molecule specific for a molecule or for a moiety present on T lymphocytes is specific for CD8. In some embodiments the binding molecule specific for a molecule or for a moiety present on T lymphocytes is specific for CD154. In some embodiments the binding molecule specific for a molecule or for a moiety present on T lymphocytes is specific for CTLA-4. In some embodiments the binding molecule specific for a molecule or for a moiety present on T lymphocytes is specific for CD62L. In some embodiments of the method according to the first aspect the binding molecule is an immunoglobulin specific for the molecule or the moiety present on T lymphocytes. In some embodiments of the method according to the first aspect the binding molecule is a proteinaceous binding molecule with immunoglobulin-like functions specific for the molecule or the moiety present on T lymphocytes.

Examples of an immunoglobulin specific for a molecule present on T lymphocytes include, but are not limited to, an anti-CD3 immunoglobulin, an anti-CD4 immunoglobulin, an anti-CD62L (L-selectin) immunoglobulin, an anti-CD154 immunoglobulin and an anti-CTLA-4 immunoglobulin. Four illustrative examples of an anti-CD3 immunoglobulin are Muromonab-CD3, Otelixizumab, Teplizumab and Visilizumab. Three illustrative examples of an anti-CD4 immunoglobulin are Clenoliximab, Keliximab and Zanolimumab. An example of an anti-CD62L (L-selectin) immunoglobulin is Aselizumab. An example of an anti-CD154 immunoglobulin is Ruplizumab. Two examples of an anti-CTLA-4 immunoglobulin are Ipilimumab and Tremelimumab.

In a method according to the first aspect the probe may for example be or include a microparticle. In a method according to the first aspect the probe may also be or include a nanoparticle such as a nanocrystal. A micro- or nanoparticle may in some embodiments include, essentially consist of or consist of a metal, a metalloid or a polymer. In some embodiments the micro- or nanoparticle is magnetic, such as paramagnetic or supermagnetic. A binding molecule is specific for T lymphocytes may be immobilised on the surface of a micro- or nanoparticle via a covalent bond or a non-covalent bond. In some embodiments a respective nanocrystal has a maximal width selected in the range from about 1 nm to about 250 nm. In some embodiments of a method according to the first aspect the probe may be or include a microbubble. A respective microbubble may in some embodiments have a maximal width selected in the range from about 1 to about 10 μm.

In a second aspect the invention relates to a probe, which is capable of specifically binding to T lymphocytes, for use in the detection of acute renal allograft rejection. The probe is detectable by ultrasound. The use includes administration of the probe to the subject. The use further includes exposure of an allotransplanted kidney of the subject to ultrasound. Further the use includes detection of the level of T lymphocytes in the kidney. Generally a detection of the level of T lymphocytes in the kidney involves the detection of signals generated by the exposure of the kidney to ultrasound. An elevated level of T lymphocytes in the kidney indicates acute renal allograft rejection.

In some embodiments of the probe for use according to the second aspect the detection of the level of T lymphocytes in the kidney is carried out by means of an imaging technique. According to a particular embodiment of the probe for use according to the first aspect the exposure of the allotransplanted kidney to ultrasound is carried out using a high intensity focused ultrasound technique. In some embodiments the detection of the level of T lymphocytes is performed via a real-time measurement.

In some embodiments of the probe for use according to the second aspect the probe has a binding molecule immobilized thereon. The binding molecule is specific for T lymphocytes. In some embodiments of the probe for use according to the second aspect the binding molecule is specific for a molecule or for a moiety present on T lymphocytes such as CD3. The binding molecule specific for a molecule or for a moiety present on T lymphocytes may also be CD4 or CD8. In some embodiments of the probe for use according to the second aspect the binding molecule is an immunoglobulin specific for the molecule or the moiety present on T lymphocytes. In some embodiments of the probe for use according to the second aspect the binding molecule is a proteinaceous binding molecule with immunoglobulin-like functions specific for the molecule or the moiety present on T lymphocytes.

In some embodiments of the probe for use according to the second aspect the probe may be or include a microparticle. The probe for use according to the second aspect may also be or include a nanoparticle such as a nanocrystal. A micro- or nanoparticle may in some embodiments include, essentially consist of or consist of a metal, a metalloid or a polymer. In some embodiments the micro- or nanoparticle is magnetic, such as paramagnetic or supermagnetic. A binding molecule is specific for T lymphocytes may be immobilised on the surface of a micro- or nanoparticle via a covalent bond or a non-covalent bond. In some embodiments such a nanocrystal has a maximal width selected in the range from about 1 nm to about 300 nm. In some embodiments of the probe for use according to the second aspect the probe may be or include a microbubble. A respective microbubble may in some embodiments have a maximal width selected in the range from about 0.1 to about 10 μm.

In some embodiments of the probe for use according to the second aspect, the use essentially consists of administration of the probe to the subject, exposure of an allotransplanted kidney of the subject to ultrasound, and detection of the level of T lymphocytes in the kidney. In one embodiment the use of the probe for use according to the second aspect consists only of administration of the probe to the subject, exposure of an allotransplanted kidney of the subject to ultrasound, and detection of the level of T lymphocytes in the kidney. In any such embodiment an elevated level of T lymphocytes in the kidney indicates acute renal allograft rejection.

In a third aspect there is provided an immunosuppressive agent for use in the treatment of renal allograft rejection. The use includes administration of the probe to the subject. The use further includes exposure of an allotransplanted kidney of the subject to ultrasound. Further the use includes detection of the level of T lymphocytes in the kidney. Generally a detection of the level of T lymphocytes in the kidney involves the detection of signals generated by the exposure of the kidney to ultrasound. If an elevated level of T lymphocytes in the kidney is detected, the use includes adapting an immunosuppressive therapy or starting an immunosuppressive therapy to the subject. If no elevated level of T lymphocytes in the kidney is detected, the use includes not adapting an immunosuppressive therapy or not starting an immunosuppressive therapy to the subject.

In some embodiments the immunosuppressive agent for use according to the third aspect includes a corticosteroid, azathioprine, mycophenolate mofetil, a calcineurin inhibitor (tacrolimus, cyclosporine), everolimus, sirolimus, an anti-lymphocyte antibody such as an anti-lymphocyte immunoglobulin (e.g., anti-thymocyte globulin, anti-lymphocyte globulin, Alemtuzumab) an antibody directed against a serum factor or a combination of any two or more of such agents. Examples of an antibody directed against a serum factor include, but are not limited to, an anti-TNFα immunoglobulin such as Adalimumab, Certolizumab pegol, Golimu-mab, Infliximab, or Nerelimomab, an Interleukin 5 immunoglobulin such as Mepolizumab, an anti-IgE immunoglobulin such as Omalizumab, an anti-interferon immunoglobulin such as Faralimomab, an anti-IL-12 and -IL-23 immunoglobulin such as Lebrikizumab or Ustekinumab, an anti-IL-6 immunoglobulin such as Elsilimomab. Examples of an anti-lymphocyte globulin include, but are not limited to, an anti-CD3 immunoglobulin such as Muromonab-CD3, Otelixizumab, Teplizumab or Visilizumab, an anti-CD4 immunoglobulin such as Clenoliximab, Keliximab or Zanolimumab, an anti-CD11a immunoglobulin such as Efalizumab, an anti-CD18 immunoglobulin such as Erlizumab), an anti-CD20 immunoglobulin such as Afutuzumab, Rituximab, Ocrelizumab or Pascolizumab, an anti-CD23 immunoglobulin such as Gomiliximab or Lumiliximab, an anti-CD40 immunoglobulin such as Teneliximab or Toralizumab, an anti-CD62L/L-selectin immunoglobulin such as Aselizumab, an anti-CD80 immunoglobulin such as Galiximab, an anti-CD147/Basigin immunoglobulin such as Gavilimomab, an anti-CD154 immunoglobulin such as Ruplizumab, an anti-BLyS immunoglobulin such as Belimumab an anti-CTLA-4 immunoglobulin such as Ipilimumab or Tremelimumab, an anti-CAT immuno-globulin such as Bertilimumab, Metelimumab or Lerdelimumab, an anti-α4-integrin immunoglobulin such as Natalizumab, an anti-Interleukin-6 receptor immunoglobulin such as Tocilizumab, an anti-IL-2 receptor/CD25 immunoglobulin such as Basiliximab, Daclizumab or Inolimomab, an anti-LFA-1 immunoglobulin such as Odulimomab or an anti-CD5 immuno-globulin such as Zolimomab aritox.

In some embodiments of the immunosuppressive agent for use according to the third aspect the probe has a binding molecule immobilized thereon. The binding molecule is specific for T lymphocytes. In some embodiments of the immunosuppressive agent for use according to the third aspect the binding molecule is specific for a molecule or for a moiety present on T lymphocytes such as CD3. The binding molecule specific for a molecule or for a moiety present on T lymphocytes may also be specific for CD4 or CD8. In some embodiments a molecule or a moiety present on T lymphocytes for which the binding molecule is specific, is CD154. As two further examples, the binding molecule may be specific for CTLA-4 or CD62L, or a moiety of CTLA-4 or CD62L. In some embodiments of the immunosuppressive agent for use according to the third aspect the binding molecule is an immunoglobulin specific for the molecule or the moiety present on T lymphocytes. In some embodiments of the immunosuppressive agent for use according to the third aspect the binding molecule is a proteinaceous binding molecule with immunoglobulin-like functions specific for the molecule or the moiety present on T lymphocytes.

In some embodiments of the immunosuppressive agent for use according to the third aspect, the use essentially consists of administration of the probe to the subject, exposure of an allotransplanted kidney of the subject to ultrasound, and detection of the level of T lymphocytes in the kidney. In one embodiment the use of the probe for use according to the third aspect consists only of administration of the probe to the subject, exposure of an allotransplanted kidney of the subject to ultrasound, and detection of the level of T lymphocytes in the kidney. In any such embodiment an elevated level of T lymphocytes in the kidney indicates acute renal allograft rejection.

In a fourth aspect there is provided a probe, which is capable of specifically binding to T lymphocytes, for use in stratifying a subject that/who is undergoing immuno-suppressive therapy for alteration of the immunosuppressive therapy. The subject has received a renal allograft. The probe is detectable by ultrasound. The use includes administration of the probe to the subject. The use also includes exposure of an allotransplanted kidney of the subject to ultrasound. Further the use includes detection of the level of T lymphocytes in the kidney after exposure of the renal allograft to ultrasound. Generally a detection of the level of T lymphocytes in the kidney involves the detection of signals generated by the exposure of the kidney to ultrasound. An elevated level of T lymphocytes in the kidney indicates that the subject is in need of an alteration of the immunosuppressive therapy.

In some embodiments the probe for use according to the fourth aspect has a binding molecule immobilized thereon. The binding molecule is specific for T lymphocytes. In some embodiments of the probe for use according to the fourth aspect the binding molecule is specific for a molecule or for a moiety present on T lymphocytes such as CD3. The binding molecule specific for a molecule or for a moiety present on T lymphocytes may also be CD4 or CD8. In some embodiments the binding molecule is specific for CD154 or CD62L. In some embodiments the binding molecule is specific for CTLA-4. In some embodiments of the probe for use according to the fourth aspect the binding molecule is an immunoglobulin specific for the molecule or the moiety present on T lymphocytes. In some embodiments of the probe for use according to the fourth aspect the binding molecule is a proteinaceous binding molecule with immunoglobulin-like functions specific for the molecule or the moiety present on T lymphocytes.

In some embodiments of the probe for use according to the fourth aspect the probe may be or include a microparticle. The probe for use according to the fourth aspect may also be or include a nanoparticle such as a nanocrystal. A micro- or nanoparticle may in some embodiments include, essentially consist of or consist of a metal, a metalloid or a polymer. In some embodiments the micro- or nanoparticle is magnetic, such as paramagnetic or supermagnetic. A binding molecule is specific for T lymphocytes may be immobilised on the surface of a micro- or nanoparticle via a covalent bond or a non-covalent bond. In some embodiments such a nanocrystal has a maximal width selected in the range from about 1 nm to about 500 nm. In some embodiments of the probe for use according to the fourth aspect the probe may be or include a microbubble. A respective microbubble may in some embodiments have a maximal width selected in the range from about 1 to about 20 μm. Microbubbles are commercially available, for example from Bracco Research (http://www.contrastultrasound-modality.com/index.php, as of 15 Mar. 2013), GE Healthcare (Optison), Bayer (Levovist) or Alliance Pharmaceutical (Imagent).

In some embodiments of the probe for use according to the fourth aspect, the immunosuppressive therapy includes use of an immuosuppresive agent selected from a corticosteroid, azathioprine, mycophenolate mofetil, a calcineurin inhibitor (tacrolimus, cyclosporine), everolimus, sirolimus, an anti-lymphocyte antibody (e.g., anti-thymocyte globulin, anti-lymphocyte globulin, Alemtuzumab) or a combination of any two or more of such agents.

According to a fifth aspect, the present invention provides a method of assessing the risk of acute renal allograft rejection in a subject. The method includes administering a probe to the subject. The probe is capable of specifically binding to T lymphocytes. The probe is furthermore detectable by applying ultrasound. The method also includes exposing an allotrans-planted kidney of the subject to ultrasound. Further the method includes detecting the level of T lymphocytes in the kidney. Generally the level of T lymphocytes in the kidney is being detected by detecting signals generated by exposing the kidney to ultrasound, typically at least in part induced and/or enhanced by the probe. An elevated level of T lymphocytes in the kidney indicates an increased risk of acute renal allograft rejection.

In some embodiments the method according to the fifth aspect essentially consists of administering the probe to the subject, exposing an allotransplanted kidney of the subject to ultrasound, and detecting the level of T lymphocytes in the kidney. In one embodiment the method according to the fifth aspect consists only of administering the probe to the subject, exposing an allotransplanted kidney of the subject to ultrasound and detecting the level of T lymphocytes in the kidney. In any such embodiment an elevated level of T lymphocytes in the kidney indicates an increased risk of acute renal allograft rejection.

In some embodiments of the method according to the fifth aspect detecting the level of T lymphocytes in the kidney is carried out by means of an imaging technique. In some embodiments detecting the level of T lymphocytes is performed via a real-time measurement. According to a particular embodiment of the method according to the fifth aspect exposing the allotransplanted kidney to ultrasound is carried out using a high intensity focused ultrasound technique.

In some embodiments of the method according to the fifth aspect the probe has a binding molecule immobilized thereon. The binding molecule is specific for T lymphocytes. In some embodiments of the method according to the fifth aspect the binding molecule is specific for a molecule or for a moiety present on T lymphocytes such as CD3. The binding molecule specific for a molecule or for a moiety present on T lymphocytes may also be specific for CD4. In some embodiments the binding molecule specific for a molecule or for a moiety present on T lymphocytes is specific for CD8. In some embodiments the binding molecule specific for a molecule or for a moiety present on T lymphocytes is specific for CD154. In some embodiments the binding molecule specific for a molecule or for a moiety present on T lymphocytes is specific for CTLA-4. In some embodiments the binding molecule specific for a molecule or for a moiety present on T lymphocytes is specific for CD62L. In some embodiments of the method according to the fifth aspect the binding molecule is an immunoglobulin specific for the molecule or the moiety present on T lymphocytes. In some embodiments of the method according to the fifth aspect the binding molecule is a proteinaceous binding molecule with immunoglobulin-like functions specific for the molecule or the moiety present on T lymphocytes.

In a sixth aspect there is provided a method of treating renal allograft rejection in a subject. The subject has received a renal allograft. The method includes administering a probe to the subject. The probe is capable of specifically binding to T lymphocytes. The probe is furthermore detectable by ultrasound. The method also includes exposing an allotransplanted kidney of the subject to ultrasound. Furthermore the method includes detecting the level of T lymphocytes in the kidney.

In some embodiments the method according to the sixth aspect essentially consists of administering the probe to the subject, exposing an allotransplanted kidney of the subject to ultrasound, and detecting the level of T lymphocytes in the kidney. In some embodiments the method according to the sixth aspect essentially consists of administering the probe to the subject, exposing an allotransplanted kidney of the subject to ultrasound, and detecting the level of T lymphocytes in the kidney.

In some embodiments of the method according to the sixth aspect, starting an immunosuppressive therapy includes administering an immunosuppressive agent to the subject. In some embodiments adapting immunosuppressive therapy includes increasing the dose of an immunosuppressive agent that is currently administered to the subject, or administering a further or a different immunosuppressive agent to the subject. Such a further or a different immuno-suppressive agent is generally an immunosuppressive agent that has currently not been administered to the subject.

In some embodiments of the method according to the sixth aspect the probe has a binding molecule immobilized thereon. The binding molecule is specific for T lymphocytes. In some embodiments of the method according to the sixth aspect the binding molecule is specific for a molecule or for a moiety present on T lymphocytes such as CD3. The binding molecule specific for a molecule or for a moiety present on T lymphocytes may also be specific for CD4. In some embodiments the binding molecule is specific for CD8. In some embodiments the binding molecule specific for a molecule or for a moiety present on T lymphocytes is specific for CD154. In some embodiments the binding molecule is specific for CTLA-4. In some embodiments the binding molecule specific for a molecule or for a moiety present on T lymphocytes is specific for CD62L. In some embodiments of the method according to the sixth aspect the binding molecule is an immunoglobulin specific for the molecule or the moiety present on T lymphocytes. In some embodiments of the method according to the sixth aspect the binding molecule is a proteinaceous binding molecule with immunoglobulin-like functions specific for the molecule or the moiety present on T lymphocytes.

In some embodiments of a method according to the sixth aspect the probe is or includes a nanoparticle such as a nanocrystal. In some embodiments a respective nanocrystal has a maximal width selected in the range from about 1 nm to about 200 nm. In some embodiments of a method according to the sixth aspect the probe is or includes a microbubble. A respective microbubble may in some embodiments have a maximal width selected in the range from about 0.5 to about 6 μm.

### BRIEF DESCRIPTION OF THE DRAWINGS

FIG. 1 depicts a histological view (HE staining) of acute allograft rejection 4 days after allogeneic transplantation. A: control kidney; B: transplanted kidney. A strong cellular rejection in form of tubulitis and glomerulonephritis can be seen in the transplant (FIG. 1B), including distinct interstitial T cell infiltrates.

FIG. 2 shows a contrast-sonographic image of renal perfusion. FIG. 2A: homogenous renal perfusion as a control, FIG. 2B: contrast defect in ischaemia/reperfusion injury of the kidney, FIG. 2C: representation of early inflammatory reaction/reperfusion injury by means of ex vivo labelled leucocytes. Arrowheads indicate contrast signals in regions of beginning necrosis at the tip of the papilla.

FIG. 3 illustrates the incubation of microbubbles with specific antibodies against the desired target such as a Fab portion directed against CD3. The antibody may carry a construct that can bind to the microbubbles. As an example, an immunoglobulin fragment may carry a molecule that includes Streptavidin binding to the microbubbles. The microbubbles with the immobilized antibodies are being infused, bind to the target and pulsate or burst if an impulse of ultrasound is being directed onto them. Thereby a signal detectable by ultrasound is being generated.

FIG. 4 shows the acute allograft rejection in leukocyte-enhanced CD3 ultrasound.

FIG. 4A and FIG. 4B show images obtained 10 minutes after intravenous application of 30 million human T lymphocytes, and one minute after intravenous application of 100 μl anti-human CD3 (ab28071, abcam), coupled to microbubbles (Bracco) to rats. Transplants with acute allograft rejection (day 4 after transplantation), indicated by an arrow, greatly accumulate the immunoglobulin. FIG. 4A depicts an image taken before, and FIG. 4B an image taken after application. Bright signals indicate positive detection of CD3.

FIG. 4C and FIG. 4D show that a syngenic transplant without acute allograft rejection do not produce any signals. The procedure indicated for FIGS. 4A and 4B was used at day 4 after transplantation. FIG. 4C depicts an image taken before, and FIG. 4D an image taken after application of cells and immunoglobulin. The arrow indicates the syngenic transplant, the star indicates a healthy native kidney.

FIG. 4E and FIG. 4F show that a kidney with ischaemia/reperfusion injury (45 min warm ischaemia, indicated by a star) does not induce generation of signals. The procedure indicated for FIGS. 4A and 4B was used at day 4 after surgery. FIG. 4E depicts an image taken before, and FIG. 4F an image taken after application of cells and immunoglobulin.

FIG. 4G and FIG. 4H show that a kidney with acute Cyclosporin A toxicity (Cyclosporin A, 50 mg/kg for 2d intraperitoneally, indicated by a star) does not induce generation of signals. The procedure indicated for FIGS. 4A and 4B was used at day 4 after transplantation. FIG. 4E depicts an image taken before, and FIG. 4F an image taken after application of cells and immunoglobulin.

FIG. 5 depicts a comparison of the detected relative amount of T lymphocytes in acute allograft rejection (during days 1 to 4 after surgery), syngenic transplants, ischaemia/reperfusion injury and acute Cyclosporin A toxicity. Measurements were taken after application of human T lymphocytes and microbubbles with an immobilized anti-human CD3 immuno-globulin (cf. the legend of FIGS. 4a and 4B) to rats. Already two days after transplantation, transplanted allogeneic rat kidneys show a strong and significant signal of CD3 positive cells in ultrasound, whereas control groups (syngenic transplants, kidneys with ischaemia/reperfusion injury, kidneys with acute Cyclosporin A toxicity) do not show such signal. IRI: ischaemia/reperfusion injury; CytATox: Cyclosporin A toxicity; POD: postoperative day.

FIG. 6 depicts a comparison of the detected relative amount of T lymphocytes in acute allograft rejection (during days 1 to 4 after surgery), syngenic transplants, ischaemia/reperfusion injury and acute Cyclosporin A toxicity. Measurements were taken 5 minutes after microbubbles with an immobilized anti-rat CD3 immunoglobulin (ab95508, abcam, 100 μl) had been applied to rats. A significant enrichment of CD3 could be detected 4 days after allogeneic kidney transplantation in the graft with acute allograft rejection. Control groups (syngenic transplants, kidneys with ischaemia/reperfusion injury, kidneys with acute Cyclosporine toxicity) do not show such signal. IRI: ischaemia/reperfusion injury; CytATox: Cyclosporin A toxicity; POD: postoperative day.

FIG. 7 shows periodic-acid-Schiff staining (PAS) of kidneys of allogeneically kidney-transplanted rats (aTX). Typical histological signs of acute allogeneic renal transplant rejection, namely glomerulitis, tubulitis and endothelialitis, which are absent in all controls (native control, syngeneically transplanted rats [sTX], acute tubular necrosis [ATN] and acute cyclosporine A nephrotoxicity [CSA]) can be found. Congruently, a significantly elevated infiltration with CD3 positive T-cells in the interstitium was found only in renal allografts. “CD3(A)” indicates a representation of colour photos taken following immunostained with antibodies against human CD3, subunit epsilon (Abcam, Cambridge, United Kingdom) and alkaline phosphatase with antibodies against CD3 epsilon (Thermoscientific, Bonn, Germany) and counterstaining with Haemalaun. “CD3(B)” indicates an edited representation of the “CD3(A)” colour photos, in which the red colour has been turned to black and the intensities of green and blue have been lightened in order to enhance the antibody staining.

FIG. 8: Analysis of flow cytometry confirmed a T-lymphocyte purity >90% (n=3). Approximately 60% of the isolated cells belong to the subpopulation of CD4⁺ T-helper cells, whereas about 30% are part of the group of CD8⁺ cytotoxic T-effector cells. (CD4⁺/CD8⁺ ratio: 1.9). Shown is a routine T-lymphocyte staining with antibodies against CD3, CD4, CD8 and CD45.

FIG. 9: T-cell labeling efficiency and stability. A) T-lymphocytes were labeled with ¹⁸F-FDG under different conditions and labeling efficiency was calculated as a ratio between pelleted cells and supernatant. Neither K⁺ nor insulin or the combination of both significantly influenced the labeling efficiencies (P>0.05). Mean values±SEM with number of observations indicated above the bar (N=3, 5×10⁶ cells, 7 MBq, P>0.05). B) Labeling stability was analyzed after 10, 30, 60 and 120 min respectively. Retention of the radionuclide in T-cells slowly decreased from 80.7±0.64% after 10 min to 45.2±0.97% after 120 min (n=7-8, P<0.05 for all time points).

FIG. 10: A) Representative PET images (day 4 after surgery) of dynamic whole body acquisitions over 130 min of an aTX rat after tail vein injection of 30×10⁶¹⁸F-FDG labeled T-cells (maximum intensity projection (MIP)). B) Renal activity timelines after injection of radiolabeled cells. In comparison to the native kidney, the renal allograft undergoing rejection (day 4 after surgery) starts to accumulate radiolabeled cells, reaching significance as early as 30-50 min after injection of radiolabeled T-cells and achieving a maximum after 120 min (n=3). * P<0.05 vs. native control kidney.

FIG. 11: A) Representative PET images (day 4 after surgery) of dynamic whole body acquisitions of aTX and sTX rats, rats with ATN and rats with acute CSA toxicity. Effects are summarized after tail vein injection of 30×10⁶¹⁸F-FDG labeled T-cells (MIP, whole-body acquisition for 20 min at 60 min (50-70 min p.i.) and 120 min (110-130 min p.i.) p.i.). B) Accumulation of radiolabeled T-lymphocytes was calculated as percentage of injected dose (% ID±SEM) in kidneys with acute rejection (aTX) and controls. On POD4 aTX kidneys exhibited a significantly elevated ¹⁸F-FDG-uptake in comparison to native controls (P<0.0001). The accumulation of labeled cells in kidneys with IRI or acute CSA toxicity as well as sTX was not significantly different to native controls (all groups n=5-17). Please note that the renal pelvis can contain eliminated free ¹⁸F-FDG. Therefore, it was excluded from the measurements. Moreover, in sTX, IRI and CSA ¹⁸F-FDG3 marked pins have been used for orientation reasons during image acquisition. Mean values±SEM with number of observations indicated above the bars. * indicates statistical significance to all other groups (P<0.05).

FIG. 12: Representative autoradiographs and histological photomicrographs 120 min after injection of 30×10⁶ radiolabeled T-cells at POD4. Mainly the renal cortex but also the medulla of allogeneic grafts showed a distinct accumulation of ¹⁸F-FDG labeled cells, whereas more or less no activity was found in native controls kidneys, sTX grafts, kidneys with ATN and CSA toxicity, respectively. Congruent with autoradiographic results, histological examination (hematoxylin-eosin stainings) showed typical signs of acute rejection (glomerulitis, tubulitisendothelialitis and graft infiltration) in renal allografts but not in any other control. Autoradiographies: cpm/mm²/MBq.

FIG. 13: Periodic-acid-Schiff staining showed typical histological signs of AR, namely glomerulitis, tubulitis and endothelialitis in aTX kidneys, which are absent in all controls (native control, sTX, ATN and CSA). Congruently, a significantly elevated infiltration with CD3 positive T-cells was found only in renal allografts. Finally, in allogeneic transplants human T-cells could be detected by immunohistochemistry. CD3 signals were absent in controls.

FIG. 14: A) Semi-automated quantification of infiltration by CD3 positive T-cells (as evaluated by light microscopy) exhibits a significant inflammation pattern in renal allografts when compared to all other controls. Mean values±SEM with number of observations indicated above the bars. * indicates statistical significance to all other groups (P<0.05). B) Correlation of accumulation of radiolabeled T-cells as % ID and of infiltrating CD3 positive T-cells in different groups of kidneys. A significant correlation of ¹⁸F-FDG signal and infiltrating leukocytes/T-cells was found (R²=0.61).

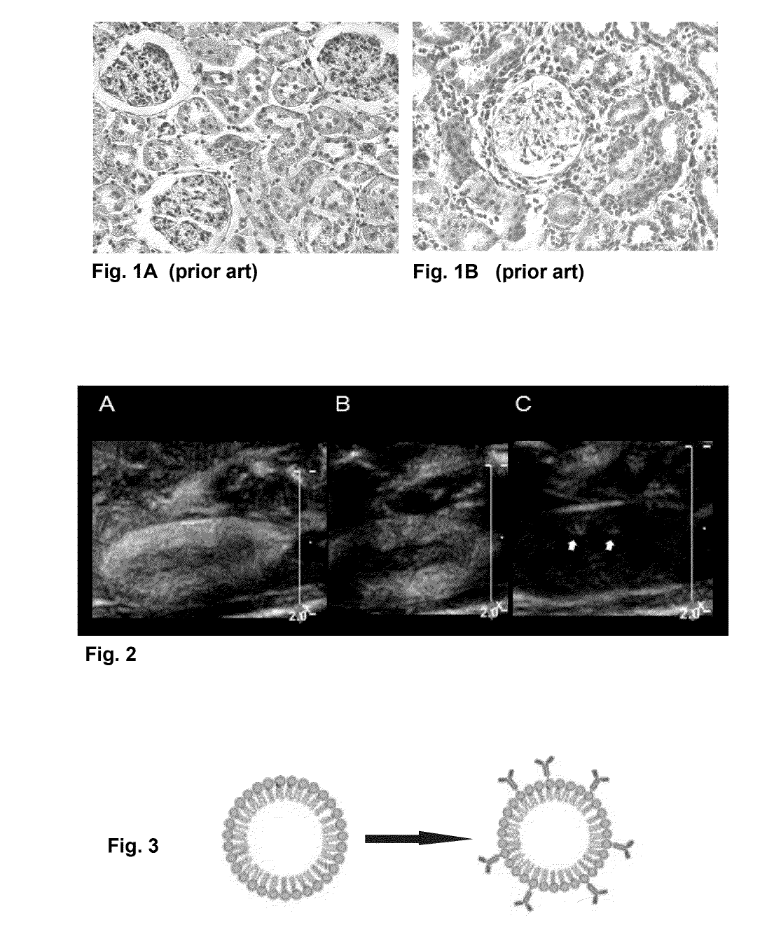

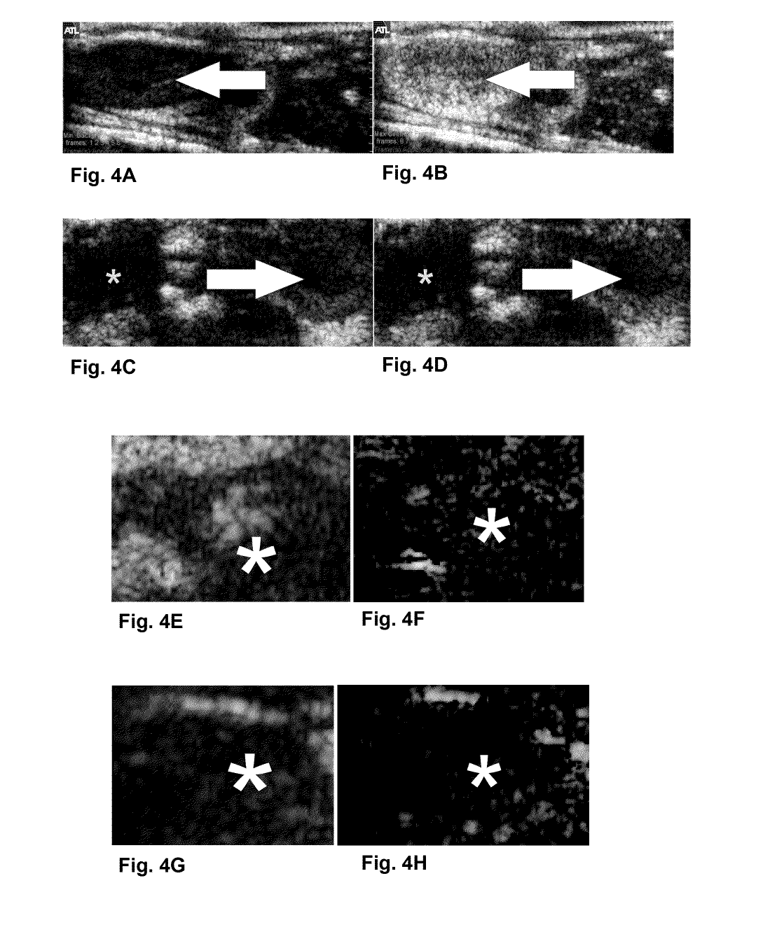

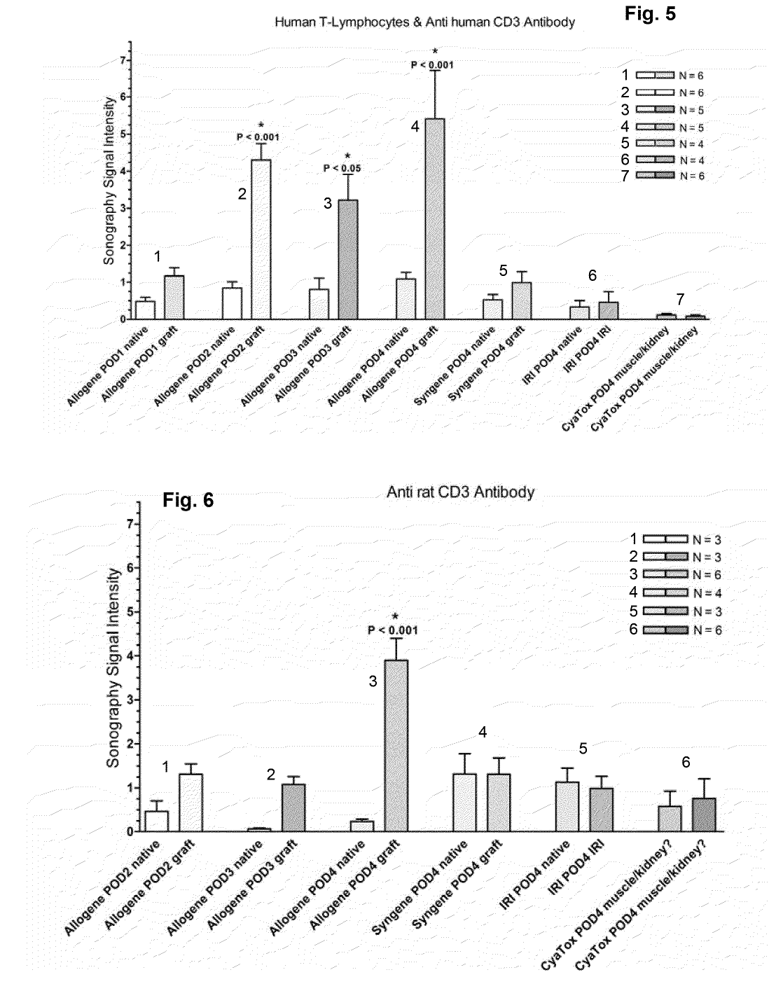

### DETAILED DESCRIPTION OF THE INVENTION

Unless otherwise stated, the following terms used in this document, including the description and claims, have the definitions given below.

The word “about” as used herein refers to a value being within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. The term “about” is also used to indicate that the amount or value in question may be the value designated or some other value that is approximately the same. The phrase is intended to convey that similar values promote equivalent results or effects according to the invention. In this context “about” may refer to a range above and/or below of up to 10%. The word “about” refers in some embodiments to a range above and below a certain value that is up to 5%, such as up to up to 2%, up to 1%, or up to 0.5% above or below that value. In one embodiment “about” refers to a range up to 0.1% above and below a given value.

The term “administering”, as used herein, refers to any mode of transferring, delivering, introducing, or transporting matter such as a compound, e.g. a pharmaceutical compound, or other agent such as an antigen, to a subject. Modes of administration include oral administration, topical contact, intravenous, intraperitoneal, intramuscular, intranasal, or subcutaneous administration (cf. below). Administration “in combination with” further matter such as one or more therapeutic agents includes simultaneous (concurrent) and consecutive administration in any order.

The term “antibody” generally refers to an immunoglobulin, a fragment thereof or a proteinaceous binding molecule with immunoglobulin-like functions (cf. below).

The word “assay” as used in this document refers to a method, generally known in the art, to analyse a feature, e.g. a catalytic activity, the presence, the formation or the amount of matter occurring in a biological specimen. Such matter may be occurring in a living organism or representing a living organism, such as a protein, a nucleic acid, a lipid, a cell, a virus, a saccharide, a polysaccharide, a vitamin or an ion, to name a few examples. The word “assay” emphasizes that a certain procedure or series of procedures is followed, which may be taken to represent the respective assay. An assay may include quantitated reagents and established protocols to assess the presence, absence, amount or activity of a biological entity.

A variety of methods for analysing binding of matter to other matter are known in the art. The techniques underlying such methods can for example be subdivided based on the use of a detectable label (cf. below). For example, some techniques require a labeled binding partner for signal detection, while others generate a signal based on the interaction of the analyte and the binding partner—including for instance measuring a mass change. Some techniques do not use labeled binding partners, but instead use a labeled analyte. Some techniques use two binding partners to create a so called “sandwich assay”, while others use only one binding partner (such as competitive assays). In sandwich assays, both binding partners bind specifically to the same analyte. In some embodiments, the two binding partners bind to differing portions, such as differing epitopes, of the analyte. Some techniques require a separation step to differentiate between a labeled binding partner that has bound an analyte and a labeled binding partner that has not bound an analyte. Some techniques do not require a separation step, such as agglutination assays and assays wherein the label on the labeled binding partner is modified, activated, or deactivated by the binding of the analyte. Some techniques require a support on which a binding partner is immobilized. A respective support may for instance be used in the context of a technique where two binding partners are employed—a first binding partner immobilized on the support, while a second binding partner is a labeled binding partner—to link the label to the support. By way of washing the support, any unbound, free labeled binding partner can then be removed prior to measuring the amount of label.

The term “binding partner” as used herein refers to matter, such as a molecule, in particular a polymeric molecule, that can bind a nucleic acid molecule such as a DNA or an RNA molecule, including an mRNA molecule, as well as a peptide, a protein, a saccharide, a polysaccharide or a lipid through an interaction that is sufficient to permit the agent to form a complex with the nucleic acid molecule, peptide, protein or saccharide, a polysaccharide or a lipid, generally via non-covalent bonding. In some embodiments the binding partner is a PNA molecule. In some embodiments the binding partner is an immunoglobulin or a proteinaceous binding molecule with immunoglobulin-like functions as defined below. In some embodiments the binding partner is an aptamer. In some embodiments a binding partner is specific for a particular target. In some embodiments a binding partner includes a plurality of binding sites, each binding site being specific for a particular target. As an illustrative example, a binding partner may be a proteinaceous agent with immunoglobulin-like functions with two binding sites. It may for instance be a bispecific diabody, such as a bispecific single chain diabody.

The term “detect” or “detecting”, as well as the term “determine” or “determining” when used in the context of a cell or a molecule present on a cell, refers to any method that can be used to perceive the presence of a nucleic acid (DNA and RNA) or a protein/polypeptide. When used herein in combination with the words “level”, “amount” or “value”, the words “detect”, “detecting”, “determine” or “determining” are understood to generally refer to a qualitative rather than a quantitative level. Nevertheless, quantification can be achieved by means of calibration using a tissue, including an artificial tissue, which includes a known number of cells with the respective molecule, e.g. T lymphocytes. Accordingly, a method disclosed in this document includes a quantification of CD3, CD4 and/or CD8—i.e. the amount or number of CD3 expressing, CD4 expressing and/or CD8 expressing T cells, e.g. CD3 positive T cells, in a selected tissue, such as a transplanted kidney, is analysed. In this regard the words “value,” “amount” and “level” are used interchangeably herein.

The term “detectable label” is used to herein to refer to any substance the detection or measurement of which, either directly or indirectly, by physical or chemical means, is indicative of the presence of a selected target bioentity in a sample. Representative examples of useful detectable labels include, but are not limited to, molecules or ions directly or indirectly detectable based on light absorbance, fluorescence, reflectivity, light scatter, phosphorescence, or luminescence properties, molecules or ions detectable by their radioactive properties or molecules or ions detectable by their nuclear magnetic resonance or paramagnetic properties. A detectable label may in some embodiments be a molecule that can be indirectly detected based on light absorbance or fluorescence, for example, various enzymes which cause appropriate substrates to convert, e.g., from non-light absorbing to light absorbing molecules, or from non-fluorescent to fluorescent molecules.

An “effective amount” or a “therapeutically effective amount” of a compound is an amount—either as a single dose or as part of a series of doses—sufficient to provide a therapeutic benefit in the treatment or management of the relevant pathological condition, or to delay or minimize one or more symptoms associated with the presence of the condition. Such a condition may be associated with immunosuppression, e.g. an autoimmune disease, or with a retroviral infection.

An “epitope” is antigenic and thus an epitope may also be taken to define an “antigenic structure” or “antigenic determinant”. Thus, a binding domain of an immunoglobulin or of a proteinaceous binding molecule with immunoglobulin-like functions is an “antigen-interaction-site”. The term “antigen-interaction-site” defines, in accordance with the present specification, a motif of a polypeptide, which is able to specifically interact with a specific antigen or a specific group of antigens, e.g. CD3 and/or CD4 in different species. This binding/interaction is also understood to define a “specific recognition”. An epitope usually consists of spatially accessible surface groupings of moieties of one or more chemical entities such as polypeptide chains or mono- or polysaccharides. Surface groupings defining an epitope may for instance be groupings of amino acids or sugar side chains. An epitope usually has specific three dimensional structural characteristics, as well as specific charge characteristics. Conformational and nonconformational epitopes are distinguished in that the binding to the former but not the latter is lost in the presence of denaturing solvents (cf. also below).

The term “epitope” also refers to a site on an antigen such as CD3, CD4 or CD8, with which an immunoglobulin, a T cell receptor or a proteinaceous binding molecule with immunoglobulin-like functions forms a complex. In some embodiments, an epitope is a site on a molecule against which an immunoglobulin or a proteinaceous binding molecule with immunoglobulin-like functions will be produced and/or to which an antibody will bind. For example, an epitope can be recognized by an immunoglobulin or a proteinaceous binding molecule with immunoglobulin-like functions. The epitope may be a “linear epitope”, which is an epitope where an amino acid primary sequence contains the epitope recognized. A linear epitope typically includes at least 3, and more usually, at least 5 amino acids in a unique sequence. A linear epitope may for example include about 8 to about 10 amino acids in a unique sequence. The epitope may also be a “conformational epitope”, which in contrast to a linear epitope, is an epitope where the primary sequence of the amino acids that includes the epitope is not the sole defining component of the epitope recognized (e.g., an epitope wherein the primary sequence of amino acids is not necessarily recognized by the antibody defining the epitope). Typically a conformational epitope includes a larger number of amino acids than a linear epitope. With regard to recognition of conformational epitopes, an immunoglobulin or a proteinaceous binding molecule with immunoglobulin-like functions recognizes a 3-dimensional structure of the antigen, such as a peptide or protein, or a fragment of a peptide or protein. As an illustrative example, when a protein molecule folds to form a three dimensional structure, certain amino acids and/or all or portions of the polypeptide backbone forming the conformational epitope become juxtaposed, allowing an antibody to recognize the epitope. Methods of determining conformation of epitopes include, but are not limited to, x-ray crystallography, 2-dimensional nuclear magnetic resonance spectroscopy, site-directed spin labeling and electron paramagnetic resonance spectroscopy.

By the use of the term “enriched” in reference to a polypeptide, a nucleic acid or a cell is meant that the specific amino acid/nucleotide sequence or cell, including cell population, constitutes a significantly higher fraction (2-5 fold) of the total amino acid sequences or nucleic acid sequence present in the sample of interest than in the natural source from which the sample was obtained. The polypeptide, a nucleic acid or a cell may also constitute a significantly higher fraction than in a normal or diseased organism or than in normal or diseased cells or in the cells from which the sequence was taken. This could be caused by preferential reduction in the amount of other amino acid/nucleotide sequences or cells present, or by a preferential increase in the amount of the specific amino acid/nucleotide sequence or cell of interest, or by a combination of the two. However, it should be noted that enriched does not imply that there are no other amino acid sequences, nucleotide sequences or cells present. The term merely defines that the relative amount of the sequence of interest has been significantly increased. The term significant here is used to indicate that the level of increase is useful to the person achieving such an increase, and generally means an increase relative to other amino acid or nucleic acid sequences of about at least 2-fold, for example at least about 5- to 10-fold or even more. The term is meant to cover only those situations in which man has intervened to increase the proportion of the desired amino acid sequence, nucleotide sequence or cell.

The term “essentially consists of” is understood to allow the presence of additional components in a sample or a composition that do not affect the properties of the sample or a composition. As an illustrative example, a pharmaceutical composition may include excipients if it essentially consists of an active ingredient.

By “fragment” in reference to a polypeptide such as an immunoglobulin or a proteinaceous binding molecule is meant any amino acid sequence present in a corresponding polypeptide, as long as it is shorter than the full length sequence and as long as it is capable of performing the function of interest of the protein—in the case of an immunoglobulin specifically binding to the desired target, e.g. antigen (CD62L, CD3, CD4 or CD8, for example). The term “immunoglobulin fragment” refers to a portion of an immunoglobulin, often the hypervariable region and portions of the surrounding heavy and light chains that displays specific binding affinity for a particular molecule. A hypervariable region is a portion of an immunoglobulin that physically binds to the polypeptide target.

The terms “immunize”, “immunization”, or “immunizing” refer to exposing the immune system of an animal to an antigen or to an epitope thereof as illustrated in more detail below. The antigen may be introduced into the animal using a desired route of administration, such as injection, inhalation or ingestion. Upon a second exposure to the same antigen, the adaptive immune response, in particular T cell and B cell responses, is enhanced.

The term “immunosuppressive agent” is synonymous to “immunosuppressant” refers to a compound (e.g., polypeptide, peptide, peptidomimetic, small organic molecule, sugar, lipid) which inhibits/reduces or prevents the development of an immunological response in an organism. Within the context of the present application the term in particular refers to a compound useful to prevent the rejection of a transplanted organ (allograft rejection). The “immunosuppressive agent” of a method disclosed in this document may be an inhibitor of T-helper cell activation, a calcinneurin inhibitor, e.g. a cyclosporin or an ascomycin, such as Cyclosporin A (NEORAL®), ISAtx-247, FK506 (tacrolimus), FK778, ABT-281 or ASM981, an IL-2 antagonist, an inhibitor of T-Lymphocyte proliferation, or an inhibitor of T-Lymphocyte migration. In some embodiments the immunosuppressive agent is cyclosporin, or FKS06, or a steroid, or an antiproliferative agent such as azathioprine, mycophenolate moefitil, or it may be a combination of such compounds.

The immunosuppressive agent may in some embodiments be a mTOR inhibitor, e.g. rapamycin or a derivative thereof, e.g. Sirolimus (RAPAMUNE®), Everolimus (Certican®), CCI779, ABT578, biolimus-7, biolimus-9, a rapalog, e.g. AP23573, azathioprine, campath 1H, a SIP receptor modulator, e.g. FTY720 or an analogue thereof, an anti IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAMO, THYMOGLOBULIN®, Brequinar Sodium, OKT4, T10B9.A-3A, 33B3.1, 15-deoxyspergualin, tresperimus, Leflunomide ARAVA®, CTLA1-Ig, anti-CD25, anti-IL2R, Basiliximab (SIMULECT®), Daclizumab (ZENAPAX®), mizorbine, methotrexate, dexamethasone, ISAtx-247, SDZ ASM 981 (pimecrolimus, Elidel®), CTLA4Ig (Abatacept), B7RP-1-fc, hul 124, BTI-322, allotrap-HLA-B270, Enlimomab, ABX-CBL, antithymocyte immunoglobulin, Medi-500, Medi-507, Alefacept or efalizumab.

In some embodiments the immunosuppressant inhibits T-cell activation by blocking the IL-2 receptor such as e.g. an anti-IL2R antibody, an anti-CD25 antibody, basiliximab or daclizumab. T-cell activation and cytokine secretion appear to play central roles in the generation and potentiation of airway inflammation in chronic asthma. Patients with severe steroid-resistant asthma have been found to have significantly higher proportions of IL-2 receptor-positive activated T cells and elevated IL-2 levels in cultures of peripheral blood leukocytes. Targeting IL-2 receptors expressed on activated T cells inhibits the cascade of immune events that leads to airway inflammation and destruction in asthma. In accordance with the present invention, it has now been found that a combination of an anti-IgE antibody with an IL-2R dependent inhibitor T-cell activation is particularly effective in the treatment or prevention of allergic responses.

The term “isolated” indicates that the cell or cells, or the peptide(s) or nucleic acid molecule(s) has/have been removed from its/their normal physiological environment, e.g. a natural source, or that a peptide or nucleic acid is synthesized. Use of the term “isolated” indicates that a naturally occurring sequence has been removed from its normal cellular (i.e., chromosomal) environment. Thus, the sequence may be in a cell-free solution or placed in a different cellular environment. An isolated cell or isolated cells may for instance be included in a different medium such as an aqueous solution than provided originally, or placed in a different physiological environment. Typically isolated cells, peptides or nucleic acid molecule(s) constitute a higher fraction of the total cells, peptides or nucleic acid molecule(s) present in their environment, e.g. solution/suspension as applicable, than in the environment from which they were taken. By “isolated” in reference to a polypeptide or nucleic acid molecule is meant a polymer of amino acids (2 or more amino acids) or nucleotides coupled to each other, including a polypeptide or nucleic acid molecule that is isolated from a natural source or that is synthesized. The term “isolated” does not imply that the sequence is the only amino acid chain or nucleotide chain present, but that it is essentially free, e.g. about 90-95% pure or more, of e.g. non-amino acid material and/or non-nucleic acid material, respectively, naturally associated with it.

Isolation of a desired population of cells may in some embodiments include general cell enrichment techniques such as centrifugation, filtration or cell chromatography. Generally, isolating or enriching a desired population of cells may be carried out according to any desired technique known in the art. In some embodiments isolation of a desired population of cells may include the use of a commercially available cell isolation kit. T cells may for instance be obtained from peripheral blood, from blood, cerebrospinal fluid, or enriched fractions thereof. T cells may for instance be obtained from peripheral blood mononuclear cells (PBMC) such as human PBMCs. In some embodiments PBMC may for instance be enriched using a standard technique based on cell density and/or cell size. As an illustrative example, PBMC may be enriched or isolated via density gradient centrifugation, for example using sucrose, dextran, Ficoll® or Percoll®. T cells may then be enriched or purified from the obtained PBMCs, for example using a commercially available T cell isolation kit such as the Dynabeads® Untouched™ Human CD4 T Cells kit available from Invitrogen or the StemSep® Human CD4+T Cell Enrichment Kit from STEMCELL Technologies Inc.

A “microparticle” is a particle of a maximal width in the range from about 500 nm and 500 μm, regardless of its structural design. A microparticle typically has a central void space surrounded by a shell and may thus be regarded as a capsule. Such a microparticle contains a fluid such as a gas or a gas-forming substance (e.g. a liquid) or another substance. A microparticle with a shell may be stabilized by a so called hard shell.

A “microbubble” is a spherical bubble, in particular an ultrasound contrast agent, encapsulating a gas and/or gas-forming substance (e.g. a liquid), and may be stabilized by a so called soft shell. The microbubble may be minimally, partially, substantially, or completely filled with the gas and/or gas-forming substance. In some embodiments a microbubble consists of a gas and/or gas-forming substance, which may contain one or more further substances. Typically a microbubble measures between 500 nm and 500 μm, in the context of a method of the present disclosure often below 10 μm.

The term “nucleic acid molecule” as used herein refers to any nucleic acid in any possible configuration, such as single stranded, double stranded or a combination thereof. Examples of nucleic acids include for instance DNA molecules, RNA molecules, analogues of the DNA or RNA generated using nucleotide analogues or using nucleic acid chemistry, locked nucleic acid molecules (LNA), protein nucleic acids molecules (PNA), alkylphosphonate and alkylphosphotriester nucleic acid molecules and tecto-RNA molecules (e.g. Liu, B., et al., J. Am. Chem. Soc. (2004) 126, 4076-4077). LNA has a modified RNA backbone with a methylene bridge between C4′ and 02′, providing the respective molecule with a higher duplex stability and nuclease resistance. Alkylphosphonate and alkylphosphotriester nucleic acid molecules can be viewed as a DNA or an RNA molecule, in which phosphate groups of the nucleic acid backbone are neutralized by exchanging the P—OH groups of the phosphate groups in the nucleic acid backbone to an alkyl and to an alkoxy group, respectively. DNA or RNA may be of genomic or synthetic origin and may be single or double stranded. Such nucleic acid can be e.g. mRNA, cRNA, synthetic RNA, genomic DNA, cDNA synthetic DNA, a copolymer of DNA and RNA, oligonucleotides, etc. A respective nucleic acid may furthermore contain non-natural nucleotide analogues and/or be linked to an affinity tag or a label.

Many nucleotide analogues are known and can be used in nucleic acids used in the methods disclosed in this specification. A nucleotide analogue is a nucleotide containing a modification at for instance the base, sugar, or phosphate moieties. As an illustrative example, a substitution of 2′-OH residues of siRNA with 2′F, 2′O-Me or 2′H residues is known to improve the in vivo stability of the respective RNA. Modifications at the base moiety may be a natural or a synthetic modification of A, C, G, and T/U, a different purine or pyrimidine base, such as uracil-5-yl, hypoxanthin-9-yl, and 2-aminoadenin-9-yl, as well as a non-purine or a non-pyrimidine nucleotide base. Other nucleotide analogues serve as universal bases. Examples of universal bases include 3-nitropyrrole and 5-nitroindole. Universal bases are able to form a base pair with any other base. Base modifications often can be combined with for example a sugar modification, such as for instance 2′-O-methoxyethyl, e.g. to achieve unique properties such as increased duplex stability.

As used in this document, the expression “pharmaceutically acceptable” refers to those active compounds, materials, compositions, carriers, and/or dosage forms which are, within the scope of sound medical judgment, suitable for use in contact with the tissues of human beings and animals without excessive toxicity, irritation, allergic response, or other problems or complications, commensurate with a reasonable benefit/risk ratio.

“Plasma” as used in this disclosure refers to acellular fluid found in blood. “Plasma” may be obtained from blood by removing whole cellular material from blood by methods known in the art such as centrifugation or filtration.

The terms “polypeptide” and “protein” refer to a polymer of amino acid residues and are not limited to a certain minimum length of the product. Where both terms are used concurrently, this twofold naming accounts for the use of both terms side by side in the art.

The term “preventing” in the medical/physiological context, i.e. in the context of a physiological state, refers to decreasing the probability that an organism contracts or develops an abnormal condition.

The term “purified” is understood to be a relative indication in comparison to the original environment of the cell, thereby representing an indication that the cell is relatively purer than in the natural environment. It therefore includes, but does not only refer to, an absolute value in the sense of absolute purity from other cells (such as a homogeneous cell population). Compared to the natural level, the level after purifying the cell will generally be at least 2-5 fold greater (e.g., in terms of cells/ml). Purification of at least one order of magnitude, such as about two or three orders, including for example about four or five orders of magnitude is expressly contemplated. It may be desired to obtain the cell at least essentially free of contamination, in particular free of other cells, at a functionally significant level, for example about 90%, about 95%, or 99% pure. With regard to a nucleic acid, peptide or a protein, the above applies mutatis mutandis. In this case purifying the nucleic acid, peptide or protein will for instance generally be at least 2-5 fold greater (e.g., in terms of mg/ml).

The word “recombinant” is used in this document to describe a nucleic acid molecule that, by virtue of its origin, manipulation, or both is not associated with all or a portion of the nucleic acid molecule with which it is associated in nature. Generally a recombinant nucleic acid molecule includes a sequence which does not naturally occur in the respective wildtype organism or cell. Typically a recombinant nucleic acid molecule is obtained by genetic engineering, usually constructed outside of a cell. Generally a recombinant nucleic acid molecule is substantially identical and/or substantial complementary to at least a portion of the corresponding nucleic acid molecule occurring in nature. A recombinant nucleic acid molecule may be of any origin, such as genomic, cDNA, mammalian, bacterial, viral, semisynthetic or synthetic origin. The term “recombinant” as used with respect to a protein/polypeptide means a polypeptide produced by expression of a recombinant polynucleotide.

The terms “screening subjects”, “screening individuals” or “screening patients” in the context of risk assessment refers to a method or process of determining if a subject/patient or a plurality of subjects/patients is or is not likely to suffer from a disease or disorder such as PML, or has or does not have an increased risk of developing a disease or disorder. “Screening compounds” and a “screening assay” means a process or method used to characterize or select compounds based upon their activity from a collection of compounds.

“Serum” as used in this disclosure, refers to components of blood that do not define a cell, such as a leukocyte, and that do not define a clotting factor. Serum includes the fraction of plasma obtained after plasma or blood is permitted to clot and the clotted fraction is removed.

The term “specific” as used in this document is understood to indicate that a binding partner is directed against, binds to, or reacts with a biomarker disclosed in the present application, such as CD4, CD8, CD3, CD154, CTLA-4 or CD62L. Thus, being directed to, binding to or reacting with includes that the binding partner specifically binds to e.g. CD4, CD8 and CD3, as applicable. The term “specifically” in this context means that the binding partner reacts with CD4, CD8 or CD3, as applicable, or/and a portion thereof, but at least essentially not with another protein. The term “another protein” includes any protein, including proteins closely related to or being homologous to e.g. CD3, CD4, CD8, CD154, CTLA-4 or CD62L against which the binding partner is directed to. The term “does not essentially bind” means that the binding partner does not have particular affinity to another protein, i.e., shows a cross-reactivity of less than about 30%, when compared to the affinity to e.g. CD4, CD8 or CD3. In some embodiments the binding partner shows a cross-reactivity of less than about 20%, such as less than about 10%. In some embodiments the binding partner shows a cross-reactivity of less than about 9, 8, or 7%, when compared to the affinity to CD4, CD8 or CD3. In some embodiments the binding partner shows a cross-reactivity of less than about 6%, such as less than about 5%, when compared to the affinity to CD4, CD8 or CD3. Whether the binding partner specifically reacts as defined herein above can easily be tested, inter alia, by comparing the reaction of a respective binding partner with CD4, with CD8 or with CD3, as applicable, and the reaction of the binding partner with (an) other protein(s). The term “specifically recognizing”, which can be used interchangeably with the terms “directed to” or “reacting with” means in the context of the present disclosure that a particular molecule, generally an immunoglobulin, an immunoglobulin fragment or a proteinaceous binding molecule with immunoglobulin-like functions is capable of specifically interacting with and/or binding to at least two, including at least three, such as at least four or even more amino acids of an epitope as defined herein. Generally the immunoglobulin or proteinaceous binding molecule can thereby form a complex with the respective epitope of e.g. CD4, CD8 or CD3. Such binding may be exemplified by the specificity of a “lock-and-key-principle”. “Specific binding” can also be determined, for example, in accordance with a Western blot, ELISA-, RIA-, ECL-, IRMA-test, FACS, IHC and a peptide scan.

The terms “stratifying” and “stratification” as used herein indicate in one aspect that individuals are assigned to groups with similar characteristics such as at a similar risk level of developing acute renal allograft rejection. As an illustrative example, individuals may be stratified into risk categories. The terms “stratifying” and “stratification” as used herein indicate in another aspect that an individual is assigned to a certain group according to characteristics matching the respective group such as a corresponding risk level of developing PML. The groups may be, for example, for testing, prescribing, suspending or abandoning any one or more of a drug, surgery, diet, exercise, or intervention. Accordingly, in some embodiments of a method or use according to the invention a subject may be stratified into a subgroup of a clinical trial of a therapy. As explained in the following, the amount of T cells in the tissue may be used for risk stratification of acute rejection.

The terms “stratifying” and “stratification” according to the instant disclosure generally include identifying subjects that require an alteration of their current or future therapy. The term includes assessing, e.g. determining, which therapy a subject likely to suffer from PML is in need of Hence, in the context of a method disclosed in the present document stratification may be based on the probability (or risk) of acute allograft rejection. A method or use described in this document may also serve in stratifying the probability of the risk of acute allograft rejection or the risk of any rejection related condition for a subject. A method of stratifying a subject for PML therapy according to the instant disclosure may include detecting the amount of determining the expression level of CD62L, PSGL-1 and/or LFA-1 as described above, and/or assessing the migratory capacity of CD45+CD49d+ immune cells of the subject. As explained above, in some embodiments on a general basis a CD62L, a PSGL-1 and/or a LFA-1 binding partner can be advantageously used to screen risk patients which are at a higher risk or have a higher predisposition to develop PML.

The term “subject” as used herein, also addressed as an individual, refers to a human or non-human animal, generally a mammal A subject may be a mammalian species such as a rabbit, a mouse, a rat, a Guinea pig, a hamster, a dog, a cat, a pig, a cow, a goat, a sheep, a horse, a monkey, an ape or a human. Thus, the methods, uses and compositions described in this document are applicable to both human and veterinary disease. As explained in more detail below, the sample has been obtained from the subject. It is thus understood that conclusions drawn from expression levels in the sample and decisions based thereon concern the subject from whom/which the subject has been taken. Further, while a subject is typically a living organism, a method or use described in this document may also be used in post-mortem analysis. Where the subject is a living human who is receiving medical care for a disease or condition, it is also addressed as a “patient”.

The term “susceptibility” as used in this document refers to the proneness of a subject towards the development of a certain state or a certain condition such as a pathological condition, including allograft rejection, or towards being less able to resist a particular state than the average individual.

The terms “treatment” and “treating” as used herein, refer to a prophylactic or preventative measure having a therapeutic effect and preventing, slowing down (lessen), or at least partially alleviating or abrogating an abnormal, including pathologic, condition in the organism of a subject. Those in need of treatment include those already with the disorder as well as those prone to having the disorder or those in whom the disorder is to be prevented (prophylaxis). Generally a treatment reduces, stabilizes, or inhibits progression of a symptom that is associated with the presence and/or progression of a disease or pathological condition. The term “administering” relates to a method of incorporating a compound into cells or tissues of a subject. The term “therapeutic effect” refers to the inhibition or activation of factors causing or contributing to the abnormal condition. A therapeutic effect relieves to some extent one or more of the symptoms of an abnormal condition or disease. The term “abnormal condition” refers to a function in the cells or tissues of an organism that deviates from their normal functions in that organism. An abnormal condition can inter alia relate to cell proliferation, cell differentiation, or cell survival.

The terms “comprising”, “including,” containing”, “having” etc. shall be read expansively or open-ended and without limitation. Singular forms such as “a”, “an” or “the” include plural references unless the context clearly indicates otherwise. Thus, for example, reference to a “vector” includes a single vector as well as a plurality of vectors, either the same—e.g. the same operon—or different. Likewise reference to a “cell” includes a single cell as well as a plurality of cells. Unless otherwise indicated, the term “at least” preceding a series of elements is to be understood to refer to every element in the series. The terms “at least one” and “at least one of” include for example, one, two, three, four, or five or more elements. It is furthermore understood that slight variations above and below a stated range can be used to achieve substantially the same results as a value within the range. Also, unless indicated otherwise, the disclosure of ranges is intended as a continuous range including every value between the minimum and maximum values.

The scope and meaning of any use of a term will be apparent from the specific context in which the term is used. Certain further definitions for selected terms used throughout this document are given in the appropriate context of the detailed description, as applicable. Unless otherwise defined, all other scientific and technical terms used in the description, figures and claims have their ordinary meaning as commonly understood by one of ordinary skill in the art.

The present invention provides, amongst others, methods of detecting acute renal allograft rejection. Using such a method a subject can be identified as currently developing allograft rejection when compared to otherwise apparently similar subjects, e.g. subjects that/who have received a kidney transplant and are of comparable health/disease state or risk factor exposure. In some embodiments a respective method can thus be taken to define a method of assessing the risk level of a subject with regard to occurrence of acute renal allograft rejection. Based on such an assessment of the risk of occurrence of acute renal allograft rejection, a decision is in some embodiments taken as to whether an immunosuppressive therapy, for example of administering cyclosporine and/or a corticosteroid, is to be continued or altered. Methods provided in this specification also allow stratifying patients for risk of acute renal allograft rejection.

In renal allografts, episodes of rejection, and in particular an acute rejection, are characterized by recruitment of activated leukocytes into the transplant. This is an integral part of the basic concept of the Banff classification, a commonly used score of renal rejection (Solez, K., et al., American Journal of Transplantation [2008] 8, 753-760).

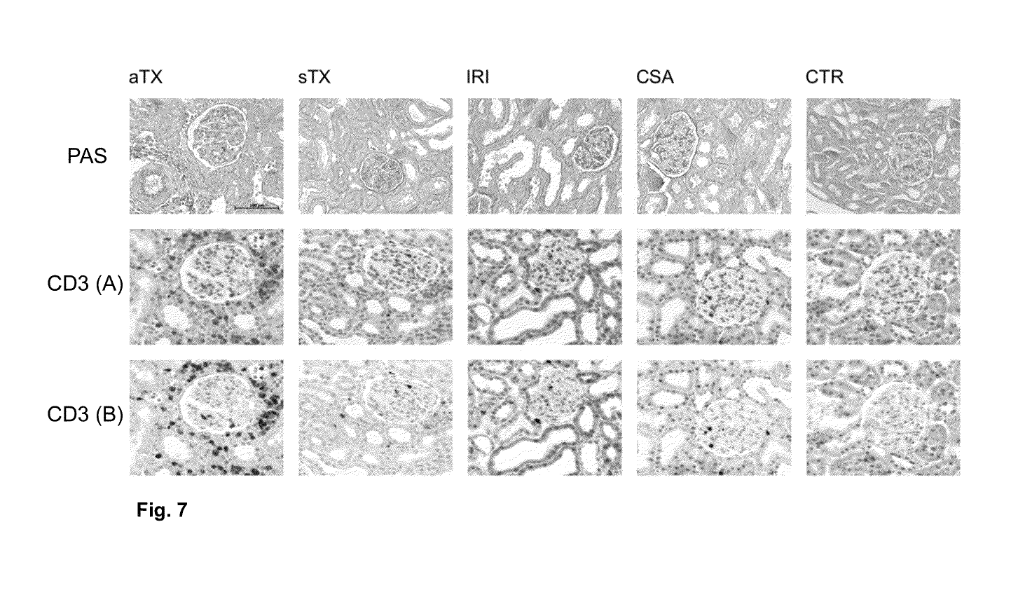

In transplantation a relatively great portion, namely 1 to 10%, of T lymphocytes is known to respond to the graft (e.g. Nankivell, B. J., and Alexander, S. I., The New England Journal of Medicine [2010] 363, 15, 1451-1462). In acute rejection of renal allografts T lymphocytes are known to infiltrate and proligerate within the interstitial space (ibid.; Racusen, L. C., et al., Kidney International [1999] 55, 713-723; see also FIG. 7). This accumulation in the interstituum of T cells, which are mainly CD4⁺ and CD8⁺ T lymphocytes, is accompanied by inflammation of the tubules and in some occasions of the arteries (Cornell, L. D., et al., Annu. Rev. Pathol. Mech. Dis. [2008] 3, 189-220). The inventors were thus surprised to find that microbubbles as well as nanoparticles, with antibodies immobilized thereon, both of which not being able to enter the interstitial space, gave a significant signal in ultrasound when located within a kidney undergoing acute renal allograft rejection.

In a method disclosed in this specification a probe is administered to a subject. Suitable routes of administration of the probe may, for example, include depot, oral, rectal, transmucosal, or intestinal administration; parenteral delivery, including an intramuscular, subcutaneous, intravenous, intramedullary injections, as well as an intrathecal, direct intraventricular, intraperitoneal, intranasal, or an intraocular injection.

The probe may be included in a pharmaceutical composition, which may be formulated in conventional manner using one or more physiologically acceptable carriers including excipients and auxiliaries that facilitate processing of the active compounds into preparations that can be used pharmaceutically. Proper formulation is dependent upon the route of administration chosen. For injection, the probe may be formulated in aqueous solutions, for instance in a physiologically compatible buffer such as Hanks's solution, Ringer's solution, or physiological saline buffer. For transmucosal administration, penetrants appropriate to the barrier to be permeated are used in the formulation. Such penetrants are generally known in the art. For oral administration, the probe can be formulated readily by combining the probe with one or more pharmaceutically acceptable carriers well known in the art. Such carriers may enable the probe to be formulated as tablets, pills, dragees, capsules, liquids, gels, syrups, slurries or suspensions, for oral ingestion by a subject.

The probe may be any matter that is capable of binding to T lymphocytes. In some embodiments the probe includes hydrocarbon-based matter. The probe may be of proteinaceous nature or include a proteinaceous portion. In some embodiments the probe is a polypeptide/protein. The probe may include a lipid, a carbohydrate, a nucleic acid, or any other polymeric or oligomeric molecule. In some embodiments the probe consists of a polymeric or oligomeric molecule such as a carbohydrate, a nucleic acid, a lipid or a combination of a carbohydrate, a lipid and a nucleic acid. The probe may also include inorganic matter such as a metal, a metalloid or a non-metal, whether in elemental form or in form of a compound. The probe is generally of dimensions that allow it to flow in the subject's blood stream. In typical embodiments the probe has dimensions that are small enough to allow the probe to enter capillary vessels in the subject's organism. In some embodiments the probe includes a nanoparticle, which may be homogenous or inhomogeneous. Any desired nanoparticle may be included in the probe. A respective nanoparticle may be of any desired shape, including ball-shaped, i.e. spherical, or rod shape. In some embodiment the nanoparticle is a nanofilament. It may for instance be a nanotube, which is hollow, or a nanowire, which is solid.

A nanoparticle used in a method described herein is typically of solid or semi-solid matter. The nanoparticle may in some embodiments be a nanosphere, i.e. an entirely filled particle without a void or cavity. In some embodiments the nanoparticle may be of non-homogenous structure. As an illustrative example, the nanoparticle may have a core and a shell, where the core has a different composition of matter than the shell. A respective shell may in some embodiments include a polymer, which may be of synthetic origin or isolated from a natural source. In some embodiments a respective polymer is an oliopeptide or a polypeptide. The nanoparticle may in some embodiments be a nanocrystal. A nanocrystal is generally of inorganic matter. In some embodiments a respective nanocrystal includes paramagnetic matter. In some embodiments a nanocrystal includes ferromagnetic matter (also termed (super-paramagnetic). Examples of ferromagnetic matter include, but are not limited to iron, nickel, and cobalt. In some embodiments the nanocrystal includes matter that can have ferromagnetic properties under certain conditions. In some embodiments the nanocrystal is at least essentially free of ferromagnetic paramagnetic matter, i.e. matter that is ferromagnetic at standard conditions (atmospheric pressure, temperature about 18° C.) including entirely free thereof. In some embodiments the nanocrystal is at least essentially free of paramagnetic matter, including entirely free thereof. Examples of paramagnetic matter include, but are not limited to magnesium, molybdenum, lithium, and tantalum. In some embodiments the nanocrystal includes diamagnetic matter.

In some embodiments the inorganic matter of a nanocrystal is or includes a semiconductor. Such a nanocrystal is typically a quantum dot. Quantum dots can be as small as 2 to 10 nanometers, with self-assembled quantum dots typically ranging between 10 and 50 nanometers in size. A nanoparticle, including a quantum dot, used in a method of this disclosure may be of any desired size, e.g. selected in the range from about 1 to about 200 nanometers, from about 1 to about 150 nanometers, from about 2 to about 150 nanometers, from about 1 to about 100 nanometers, from about 2 to about 100 nanometers, from about 1 to about 80 nanometers or from about from about 2 to about 80 nanometers.

In some embodiments the probe includes a microbubble. A respective microbubble typically has a core of a fluid and a shell that is immiscible with both the fluid of the core and with blood. The core may include a liquid and/or a gas. In some embodiments the core includes an inert gas such as a noble gas or nitrogen. The core may in some embodiments include a perfluorocarbon gas such as perfluorobutane. In some embodiments the core includes an aqueous liquid. The shell of a respective microbubble includes in some embodiments amphophilic compounds such as a fatty acid or a phospholipid or polybutylcyanoacrylate. In addition the shell may include polar matter, including semipolar matter. Polar matter is typically hydrophilic and soluble in water, but insoluble in apolar solvents such as toluene or Castor oil. According to their dielectric constant semipolar matter may be defined as having a dielectric constant in the range from about 30 to 50 at a temperature of 20° C. and atmospheric pressure, which is for instance the case for glycerol, polyethylene glycol, methanol or propylene glycol. The shell may in some embodiments include a monosaccharide, a disaccharide or a polysaccharide. In some embodiments the shell includes a polypeptide, for instance in native or in denatured form.

A microbubble that is included in a probe used in a method according to the instant document is typically spherical and has a shape that resembles a uniform ball. In some embodiments a respective microbubble has a radius in the range from about 0.1 to about 15 μm. In some embodiments the radius of such a microbubble may be selected in the range from about 1 to about 5 μm.

In some embodiments the probe may have a moiety that is capable of binding to T lymphocytes, typically a moiety that specifically binds to T lymphocytes. Such a moiety generally has a binding affinity for T lymphocytes. In some embodiments the probe may be coupled to a molecule that is capable of binding to T lymphocytes, typically a molecule that is capable of specifically binding to T lymphocytes. A respective molecule generally has a binding affinity for T lymphocytes. A molecule capable of specifically binding to T lymphocytes may include a portion that is capable of specifically binding to a moiety or a molecule found on T lymphocytes. Such a molecule capable of specifically binding to T lymphocytes generally has a binding affinity for the molecule found on T lymphocytes. An illustrative example of a suitable molecule in this regard is an antibody (supra).

In some embodiments a molecule that is capable of specifically binding to T lymphocytes, herein also abbreviated “binding molecule”, is coupled to the probe. The binding molecule may be coupled to the probe via a linking moiety such as an affinity tag. Such a linking moiety may be a molecule, e.g. a hydrocarbon-based (including polymeric) molecule that includes nitrogen-, phosphorus-, sulphur-, carben-, halogen- or pseudohalogen groups, or a portion thereof. As an illustrative example, the surface of the probe may include, for instance be coated with, a brush-like polymer, for example with short side chains. The surface of the probe may also include a polymer that includes a brush-like structure, for example by way of grafting. It may for example include functional groups that allow for the covalent attachment of a biomolecule, for example a molecule such as a protein, a nucleic acid molecule, a polysaccharide or any combination thereof. Examples of a respective functional group include, but are not limited to, an amino group, an aldehyde group, a thiol group, a carboxy group, an ester, an anhydride, a sulphonate, a sulphonate ester, an imido ester, a silyl halide, an epoxide, an aziridine, a phosphoramidite and a diazoalkane.

Examples of an affinity tag include, but are not limited to biotin, dinitrophenol or digoxigenin, oligohistidine, polyhistidine, an immunoglobulin domain, maltose-binding protein, glutathione-S-transferase (GST), calmodulin binding peptide (CBP), FLAG′-peptide, the T7 epitope (Ala-Ser-Met-Thr-Gly-Gly-Gln-Gln-Met-Gly) (SEQ ID No. 1), maltose binding protein (MBP), the HSV epitope of the sequence Gln-Pro-Glu-Leu-Ala-Pro-Glu-Asp-Pro-Glu-Asp (SEQ ID No. 2) of herpes simplex virus glycoprotein D, the hemagglutinin (HA) epitope of the sequence Tyr-Pro-Tyr-Asp-Val-Pro-Asp-Tyr-Ala (SEQ ID No. 3), the “myc” epitope of the transcription factor c-myc of the sequence Glu-Gln-Lys-Leu-Ile-Ser-Glu-Glu-Asp-Leu (SEQ ID No. 4), or an oligonucleotide tag. Such an oligonucleotide tag may for instance be used to hybridise to an immobilised oligonucleotide with a complementary sequence. A further example of a linking moiety is an immunoglobulin, a fragment of an immunoglobulin or a proteinaceous binding molecule with immunoglobulin-like functions (see also above).

A further example of a linking moiety is a cucurbituril or a moiety capable of forming a complex with a cucurbituril. A cucurbituril is a macrocyclic compound that includes glycoluril units, typically self-assembled from an acid catalyzed condensation reaction of glycoluril and formaldehyde. A cucurbit[n]uril, (CB[n]), that includes n glycoluril units, typically has two portals with polar ureido carbonyl groups. Via these ureido carbonyl groups cucurbiturils can bind ions and molecules of interest. As an illustrative example cucurbit[7]uril (CB[7]) can form a strong complex with ferrocenemethylammonium or adamantylammonium ions. Either the cucurbit[7]uril or e.g. ferrocenemethylammonium may be attached to a biomolecule, while the remaining binding partner (e.g. ferrocenemethylammonium or cucurbit[7]uril respectively) can be bound to a selected surface. Contacting the biomolecule with the surface will then lead to an immobilisation of the biomolecule. Functionalised CB[7] units bound to a gold surface via alkanethiolates have for instance been shown to cause an immobilisation of a protein carrying a ferrocenemethylammonium unit (Hwang, I., et al., J. Am. Chem. Soc. (2007) 129, 4170-4171).

Further examples of a linking moiety include, but are not limited to, an oligosaccharide, an oligopeptide, biotin, dinitrophenol, digoxigenin and a metal chelator (cf. also below). As an illustrative example, a respective metal chelator, such as ethylenediamine, ethylenediaminetetraacetic acid (EDTA), ethylene glycol tetraacetic acid (EGTA), diethylenetri-aminepentaacetic acid (DTPA), N,N-bis(carboxymethyl)glycine (also called nitrilotriacetic acid, NTA), 1,2-bis(o-aminophenoxy)ethane-N,N,N′,N′-tetraacetic acid (BAPTA), 2,3-dimercapto-1-propanol (dimercaprol), porphine or heme may be used in cases where the target molecule is a metal ion. As an example, EDTA forms a complex with most monovalent, divalent, trivalent and tetravalent metal ions, such as e.g. silver (Ag⁺), calcium (Ca²⁺), manganese (Mn²⁺), copper (Cu²⁺), iron (Fe²⁺), cobalt (Co³⁺) and zirconium (Zr⁴⁺), while BAPTA is specific for Ca²⁺. In some embodiments a respective metal chelator in a complex with a respective metal ion or metal ions defines the linking moiety. Such a complex is for example a receptor molecule for a peptide of a defined sequence, which may also be included in a protein. As an illustrative example, a standard method used in the art is the formation of a complex between an oligohistidine tag and copper (Cu²⁺), nickel (Ni²⁺), cobalt (Co²⁺), or zink (Zn²⁺) ions, which are presented by means of the chelator nitrilotriacetic acid (NTA).

Avidin or streptavidin may for instance be employed to immobilise a biotinylated peptide or nucleic acid, or a biotin containing monolayer of gold may be employed (Shumaker-Parry, J. S., et al., Anal. Chem. (2004) 76, 918). As an illustrative example, in some embodiments the probe surface may include one or more streptavidin molecules. A respective streptavidin molecule is a binding partner for biotin. A biotinylated antibody may be immobilized onto the surface of the probe via binding to streptavidin. As yet another illustrative example, a binding molecule may be locally deposited on a surface of a probe, e.g. by scanning electrochemical microscopy, for instance via pyrrole-oligonucleotide patterns (e.g. Fortin, E., et al., Electroanalysis (2005) 17, 495). In other embodiments, in particular where the binding molecule is a nucleic acid, the biomolecule may be directly synthesised on the surface of the probe, for example using photoactivation and deactivation. As an illustrative example, the synthesis of nucleic acids or oligonucleotides on selected surface areas (so called “solid phase” synthesis) may be carried out using electrochemical reactions using electrodes. An electrochemical deblocking step as described by Egeland & Southern (Nucleic Acids Research (2005) 33, 14, e125) may for instance be employed for this purpose. A suitable electrochemical synthesis has also been disclosed in US patent application US 2006/0275927. In some embodiments light-directed synthesis of a binding molecule, in particular of a nucleic acid molecule, including UV-linking or light dependent 5′-deprotection, may be carried out.

The molecule that has a binding affinity for T cells, including for a selected target molecule on the surface of T cells, may be immobilised on the probe, e.g. the nanocrystal, by any means. As an illustrative example, an oligo- or polypeptide, including a respective moiety, may be covalently linked to the surface of nanocrystals via a thio-ether-bond, for example by using w functionalized thiols. Any suitable molecule that is capable of linking a nanocrystal of the invention to a molecule having a selected binding affinity may be used to immobilise the same on a nanocrystal. For instance a (bifunctional) linking agent such as ethyl-3-dimethylaminocarbodiimide, N-(3-aminopropyl) 3-mercapto-benzamide, 3-aminopropyl-trimethoxysilane, 3-mercaptopropyl-trimethoxysilane, 3-(trimethoxysilyl) propyl-maleimide, or 3-(trimethoxysilyl) propyl-hydrazide may be used. Prior to reaction with the linking agent, the surface of the nanocrystals can be modified, for example by treatment with glacial mercaptoacetic acid, in order to generate free mercaptoacetic groups which can then employed for covalently coupling with an analyte binding partner via one or more linking agents.

T cells are known to the skilled artisan as lymphocytes, i.e. nucleated blood cells that are also called white blood cells. T cells mature in the thymus and can be distinguished from other lymphocytes in that they have the T cell receptor on their cell surface. The main known role of the T cell is recognition of antigens bound to major histocompatibility complex (MHC) molecules. The T cell receptor (TCR) is a heterodimer, which in about 95% of T cells consists of a 34 kD α-chain, linked by a disulphide bond to a 34 kD β-chain. Both chains span the plasma membrane and have accordingly an extracellular portion, each of which includes a variable region, termed Vα and Vβ, respectively. About 5% of T cells have a T cell receptor that consists of a γ- and a δ-chain instead of an α- and a β-chain, which likewise have extracellular variable regions. T cell receptors can, like immunoglobulins, recognize a very large number of different epitopes.

A molecule found on T lymphocytes to which a probe, including a binding moiety or a binding molecule included in or coupled to the probe, may bind, is thus in some embodiments the T cell receptor on the surface of a T cell. As the T cell receptor has variable regions it may in some embodiments be advantageous to use another cell surface protein, whether in addition to or instead of the T cell receptor, as a target for specific binding, i.e. to identify a T cell. An example of a suitable protein in this regard is a T cell co-receptor. Two illustrative examples of a co-receptor of the T cell receptor are the protein complex CD3 (Cluster of Differentiation 3) and the protein CD247. CD3 has four chains, which are in mammals one D3γ chain, one CD3δ chain, and two CD3∈ chains. These chains associate with a molecule known as the T-cell receptor and at least one T-cell surface glycoprotein CD3 zeta chain also known as T-cell receptor T3 zeta chain or CD247 (Cluster of Differentiation 247). CD247 may be present on the cell surface as either a ζ₂ complex or a ζ/η complex. The complex of TCR, CD247 and CD can generate an activation signal in T lymphocytes. The TCR, ζ-chain(s), and CD3 molecule together define the TCR complex. In practicing a method according to the invention identifying the presence of CD3 on a particular cell or plurality of cells is often a convenient way of identifying T cells. Therefore, the terms “CD3⁺ T cell” and “T cell” can generally be used interchangeable to address a T cell and to distinguish a T cell from other cell types.

A further example of a co-receptor of the T cell receptor, present on some but not all T cells, is the transmembrane protein CD8 (Cluster of Differentiation 8). Most T cells that have CD8 on their surface are cytotoxic T cells. CD8 plays an important role in binding to the class I major histocompatibility complex. Two isoforms of the protein, namely CD8-alpha and -beta, are known. Each such chain contains a domain that resembles an immunoglobulin variable domain. CD8 is a dimer of two of these chains, either a homo- or a heterodimer.

CD4⁺ T cells have, generally in addition to CD3, the CD4 (Cluster of Differentiation 4) protein on their surface, a glycoprotein consisting of four extracellular immunoglobulin domains, termed D₁ to D₄, and a small cytoplasmic region. The CD4 protein is known to be used by HIV-1 to gain entry into T cells of a host. CD4⁺ T cells can be classified into a variety of cell populations with different functions and should thus not be taken to define a unitary set of cells. Typical examples of a CD4⁺ T cell are a T helper cell, a regulatory T cell and a memory T cell.

In some embodiments a T lymphocyte to which the probe specifically binds is a CD3⁺ T cell. A T lymphocyte to which the probe specifically binds may in some embodiments be a CD4⁺ T cell. In some embodiments a T lymphocyte to which the probe specifically binds is a CD8⁺ T cell. In some embodiments a T lymphocyte to which the probe specifically binds is a CD154⁺ T cell. A T lymphocyte to which the probe specifically binds may also be a CTLA-4⁺ T cell. In some embodiments a T lymphocyte to which the probe specifically binds is a CD62L⁺ T cell.

A binding molecule that specifically binds to a molecule, or a moiety of a molecule, such as CD3, CD4, CD8, CD154 or CD62L, as well as a binding molecule with affinity for another selected cell-characteristic protein, may be an immunoglobulin, a fragment thereof or a proteinaceous binding molecule with immunoglobulin-like functions. An antibody fragment generally contains an antigen binding or variable region. Examples of (recombinant) antibody fragments are immunoglobulin fragments such as Fab fragments, Fab′ fragments, Fv fragments, single-chain Fv fragments (scFv), diabodies or domain antibodies (Holt, L. J., et al., Trends Biotechnol. (2003), 21, 11, 484-490). An example of a proteinaceous binding molecule with immunoglobulin-like functions is a mutein based on a polypeptide of the lipocalin family (WO 03/029462, Beste et al., Proc. Natl. Acad. Sci. USA (1999) 96, 1898-1903). Lipocalins, such as the bilin binding protein, the human neutrophil gelatinase-associated lipocalin, human Apolipoprotein D or glycodelin, possess natural ligand-binding sites that can be modified so that they bind to selected small protein regions known as haptens. Examples of other proteinaceous binding molecules are the so-called glubodies (see e.g. international patent application WO 96/23879 or Napolitano, E. W., et al., Chemistry & Biology (1996) 3, 5, 359-367), proteins based on the ankyrin scaffold (Mosavi, L. K., et al., Protein Science (2004) 13, 6, 1435-1448) or crystalline scaffold (e.g. internation patent application WO 01/04144), the proteins described in Skerra, J. Mol. Recognit. (2000) 13, 167-187, AdNectins, tetranectins and avimers. Avimers contain so called A-domains that occur as strings of multiple domains in several cell surface receptors (Silverman, J., et al., Nature Biotechnology (2005) 23, 1556-1561). Adnectins, derived from a domain of human fibronectin, contain three loops that can be engineered for immunoglobulin-like binding to targets (Gill, D. S. & Damle, N. K., Current Opinion in Biotechnology (2006) 17, 653-658). Tetranectins, derived from the respective human homotrimeric protein, likewise contain loop regions in a C-type lectin domain that can be engineered for desired binding (ibid.). Peptoids, which can act as protein ligands, are oligo(N-alkyl) glycines that differ from peptides in that the side chain is connected to the amide nitrogen rather than the a carbon atom. Peptoids are typically resistant to proteases and other modifying enzymes and can have a much higher cell permeability than peptides (see e.g. Kwon, Y.-U., and Kodadek, T., J. Am. Chem. Soc. (2007) 129, 1508-1509). A suitable antibody may in some embodiments also be a multispecific antibody that includes several immunoglobulin fragments.

An immunoglobulin or a proteinaceous binding molecule with immunoglobulin-like functions may be PEGylated or hyperglycosylated if desired. In some embodiments a proteinaceous binding molecule with immunoglobulin-like functions is a fusion protein of one of the exemplary proteinaceous binding molecules above and an albumin-binding domain, for instance an albumin-binding domain of streptococcal protein G. In some embodiments a proteinaceous binding molecule with immunoglobulin-like functions is a fusion protein of an immunoglobulin fragment, such as a single-chain diabody, and an immunoglobulin binding domain, for instance a bacterial immunoglobulin binding domain. As an illustrative example, a single-chain diabody may be fused to domain B of staphylococcal protein A as described by Unverdorben et al. (Protein Engineering, Design & Selection [2012] 25, 81-88).

An immunoglobulin may be monoclonal or polyclonal. The term “polyclonal” refers to immunoglobulins that are heterogenous populations of immunoglobulin molecules derived from the sera of animals immunized with an antigen or an antigenic functional derivative thereof. For the production of polyclonal immunoglobulins, one or more of various host animals may be immunized by injection with the antigen. Various adjuvants may be used to increase the immunological response, depending on the host species. “Monoclonal immunoglobulins”, also called “monoclonal antibodies”, are substantially homogenous populations of immunoglobulins to a particular antigen. They may be obtained by any technique which provides for the production of immunoglobulin molecules by continuous cell lines in culture. Monoclonal immunoglobulins may be obtained by methods well known to those skilled in the art (see for example, Köhler et al., Nature (1975) 256, 495-497, and U.S. Pat. No. 4,376,110). An immunoglobulin or immunoglobulin fragment with specific binding affinity only for e.g. CD3, CD8, CD4 or CTLA-4 can be isolated, enriched, or purified from a prokaryotic or eukaryotic organism. Routine methods known to those skilled in the art enable production of both immunoglobulins or immunoglobulin fragments and proteinaceous binding molecules with immunoglobulin-like functions, in both prokaryotic and eukaryotic organisms.

In more detail, an immunoglobulin may be isolated by comparing its binding affinity to a protein of interest, e.g. L-selectin (CD62L), with its binding affinity to other polypeptides. Humanized forms of the antibodies of the present invention may be generated using one of the procedures known in the art such as chimerization or CDR grafting. In general, techniques for preparing monoclonal antibodies and hybridomas are well known in the art. Any animal such as a goat, a mouse, a rat or a rabbit that is known to produce antibodies can be immunized with the selected polypeptide, e.g. L-selectin. Methods for immunization are well known in the art. Such methods include subcutaneous or intraperitoneal injection of the polypeptide. One skilled in the art will recognize that the amount of polypeptide used for immunization and the immunization regimen will vary based on the animal which is immunized, including the species of mammal immunized, its immune status and the body weight of the mammal, as well as the antigenicity of the polypeptide and the site of injection.

The polypeptide may be modified or administered in an adjuvant in order to increase the peptide antigenicity. Methods of increasing the antigenicity of a polypeptide are well known in the art. Such procedures include coupling the antigen with a heterologous protein (such as globulin or β-galactosidase) or through the inclusion of an adjuvant during immunization.

Typically, the immunized mammals are bled and the serum from each blood sample is analysed for particular antibodies using appropriate screening assays. As an illustrative example, anti-CD3, anti-CD4 or anti-CD8 immunoglobulins may be identified by immunoprecipitation of ¹²⁵I-labeled cell lysates from CD3, CD8 or CD4-expressing cells. Anti-CD3, CD8 or anti-CD4 immunoglobulins may also be identified by flow cytometry, e.g., by measuring fluorescent staining of Ramos cells incubated with an immunoglobulin believed to recognize CD3, CD8 or CD4, as applicable.

For monoclonal immunoglobulins, lymphocytes, typically splenocytes, from the immunized animals are removed, fused with an immortal cell line, typically myeloma cells, such as SP2/0-Ag14 myeloma cells, and allowed to become monoclonal immunoglobulin producing hybridoma cells. Typically, the immortal cell line such as a myeloma cell line is derived from the same mammalian species as the lymphocytes. Illustrative immortal cell lines are mouse myeloma cell lines that are sensitive to culture medium containing hypoxanthine, aminopterin and thymidine (“HAT medium”). Typically, HAT-sensitive mouse myeloma cells are fused to mouse splenocytes using 1500 molecular weight polyethylene glycol (“PEG 1500”). Hybridoma cells resulting from the fusion may then be selected using HAT medium, which kills unfused and unproductively fused myeloma cells (unfused splenocytes die after several days because they are not transformed).

Any one of a number of methods well known in the art can be used to identify a hybridoma cell which produces an immunoglobulin with the desired characteristics. Typically the culture supernatants of the hybridoma cells are screened for immunoglobulins against the antigen. Suitable methods include, but are not limited to, screening the hybridomas with an ELISA assay, Western blot analysis, or radioimmunoassay. Hybridomas prepared to produce anti-CD3, anti-CD4, anti-CD8 or CD154 immunoglobulins may for instance be screened by testing the hybridoma culture supernatant for secreted antibodies having the ability to bind to a recombinant CD3, CD8, CD4 or CD154 expressing cell line. To produce antibody homologs which are within the scope of the invention, including for example, anti-CD3, -CD4, anti-CD8 or anti-CD154 antibody homologs, that are intact immunoglobulins, hybridoma cells that tested positive in such screening assays can be cultured in a nutrient medium under conditions and for a time sufficient to allow the hybridoma cells to secrete the monoclonal immunoglobulins into the culture medium. Tissue culture techniques and culture media suitable for hybridoma cells are well known in the art. The conditioned hybridoma culture supernatant may be collected and for instance the anti-CD3 immunoglobulins or the anti-CD62L immunoglobulins optionally further purified by well-known methods. Alternatively, the desired immunoglobulins may be produced by injecting the hybridoma cells into the peritoneal cavity of an unimmunized mouse. The hybridoma cells proliferate in the peritoneal cavity, secreting the immunoglobulin which accumulates as ascites fluid. The immunoglobulin may be harvested by withdrawing the ascites fluid from the peritoneal cavity with a syringe.

Hybridomas secreting the desired immunoglobulins are cloned and the class and subclass are determined using procedures known in the art. For polyclonal immunoglobulins, an immunoglobulin containing antiserum is isolated from the immunized animal and is screened for the presence of immunoglobulins with the desired specificity using one of the above-described procedures. The above-described antibodies, including immunoglobulins, may also be immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art.

A plurality of conventional display technologies is available to select an immunoglobulin, immunoglobulin fragment or proteinaceous binding molecule. Li et al. (Organic & Biomolecular Chemistry (2006), 4, 3420-3426) have for example demonstrated how a single-chain Fv fragment capable of forming a complex with a selected DNA adapter can be obtained using phage display. Display techniques for instance allow the generation of engineered immunoglobulins and ligands with high affinities for a selected target molecule. It is thus also possible to display an array of peptides or proteins that differ only slightly, typically by way of genetic engineering. Thereby it is possible to screen and subsequently evolve proteins or peptides in terms of properties of interaction and biophysical parameters. Iterative rounds of mutation and selection can be applied on an in vitro basis.

In vitro display technology for the selection of peptides and proteins relies on a physical linkage between the peptide or protein and a nucleic acid encoding the same. A large panel of techniques has been established for this purpose, with the most commonly used being phage/virus display, ribosome display, cell-surface display, ‘peptides on plasmids’, mRNA display, DNA display, and in vitro compartmentalisation including micro-bead display (for reviews see e.g. Rothe, A., et al., FASEB J. (2006) 20, 1599-1610; Sergeeva, A., et al., Advanced Drug Delivery Reviews (2006) 58, 1622-1654).

Different means of physically linking a peptide, including a protein, and a nucleic acid are also available. Expression in a cell with a cell surface molecule, expression as a fusion polypeptide with a viral/phage coat protein, a stabilised in vitro complex of an RNA molecule, the ribosome and the respective polypeptide, covalent coupling in vitro via a puromycin molecule or via micro-beads are examples of ways of linking the protein/peptide and the nucleic acid presently used in the art. A further display technique relies on a water-in-oil emulsion. The water droplets serve as compartments in each of which a single gene is transcribed and translated (Tawfik, D. S., & Griffiths, A. D., Nature Biotech. (1998) 16, 652-656, US patent application 2007/0105117). This physical linkage between the peptide including the protein, and the nucleic acid (encoding it) provides the possibility of recovering the nucleic acid encoding the selected peptide/protein. Compared to techniques such as immuno-precipitation, in display techniques thus not only binding partners of a selected target molecule can be identified or selected, but the nucleic acid of this binding partner can be recovered and used for further processing. Present display techniques thus provide means for e.g. target discovery, lead discovery and lead optimisation. Vast libraries of peptides or proteins, e.g. antibodies, potentially can be screened on a large scale.

In some embodiments of a method or use disclosed in this document, the probe has coupled to it a plurality, such as two, three or more, molecules that have a binding affinity for T cells, including for a selected target molecule on the surface of T cells. In some embodiments one or more of this plurality of binding molecules is/are an immunoglobulin. In some embodiments one or more of this plurality of binding molecules is/are a proteinaceous binding molecule with immunoglobulin-like functions. In one embodiment all binding molecules of this plurality of binding molecules are immunoglobulins. In one embodiment all binding molecules of this plurality of binding molecules are proteinaceous binding molecules with immunoglobulin-like functions.

In a method disclosed herein a transplanted kidney of the subject is being exposed to ultrasound. Any desired technique for ultrasound generation, as well as for irradiation with ultrasound, may be used as long as the ultrasound energy is sufficient to allow detection of the Ultrasound energy may be applied in a pulsed or continuous form. Those skilled in the art will be aware that ultrasonic energy can be focused to produce necrosis in living tissue. While such applications are used for treating a tumour, in the context of detection as in a method or use of the invention it may be advantageous to avoid tissue damage or destruction. Ultrasound generation may be carried out according to any established detection technique. The skilled person can choose from a variety of commercially available systems, since ultrasound imaging is the most widely used medical imaging technique. The best known medical imaging application is the visualisation of a foetus.

In some embodiments sonic energy is applied to the transplanted kidney by means of High-Intensity Focused Ultrasound. This technique is well established in the art and can be used to achieve a very small focus deep in tissues. Using this technique a selected region can be visualized. In the context of imaging lower intensities of ultrasound are used than for therapeutic purposes such as tissue ablation and hemostasis. If a hyperecho should appear, the application of ultrasound is generally discontinued in order to avoid tissue damage.

In a method described in this specification the level of T cells in the kidney is being detected. In a method or use disclosed in this document detection of leukocytes may in some embodiments be achieved if ultrasound is applied to a transplanted kidney or a portion thereof. In such embodiments the detection of leukocytes may be carried out according to any desired imaging technique. Imaging on the basis of ultrasound relies on the reflection of the transmitted sound wave at interfaces between matter of different properties, in particular matter of different density. In order to enhance the visibility of interfaces, in particular interfaces within the blood stream or in fluid communication with the blood stream, ultrasound contrast agents may be applied, typically by injection into the blood stream. The probe used in methods disclosed herein, for example in form of a microbubble or a nanoparticle, may in some embodiments be taken to define an ultrasound contrast agent. The selection of the frequency of ultrasound used for medical imaging is within the skill of those skilled in the art. Generally, the frequency is selected to be in the Megahertz range (1-50 MHz). The ultrasound imaging frequency used is typically selected according to the desired resolution and the desired imaging depth. On a general basis, the shorter the wavelength the higher the resolution and the longer the wavelength the further the penetration depth.

In some embodiments where the probe is a microbubble, the respective microbubble has a radius of about 1 to about 5 μm. Such a microbubble has a resonance frequency in the megahertz range, used in most current ultrasound imaging techniques. A microbubble will typically reflect ultrasound more efficiently than tissue, as it has a larger difference in density and speed of sound, and thus acoustic impedance, with its surroundings. In addition, a microbubble possesses a compressibility, which will cause it to undergo radial pulsations in response to an oscillating pressure field. As a result, a secondary sound wave, generated by the microbubble, is formed. In some embodiments a microbubble may also have a radius in the range from about 1 to about 4 μm. In some embodiments a microbubble is selected to have a radius that is below the average radius of erythrocytes of the subject's species. As an illustrative example, a typical human erythrocyte has a disk radius of approximately 3.1-4.1 μm. Mouse and rat erythrocytes have been found to have a radius of about 3.0 μm and 3.2 μm, respectively.

In some embodiments where the probe is a nano bubble, the respective nano bubble has a radius in the range from about 100 nm to about 1 μm. A respective nano bubble or microbubble may be an encapsulated gas bubble with a binding molecule on the shell surface. The binding molecule is typically specific for a molecule or moiety characteristic for T cells and found on the surface of T cells. An encapsulated gas bubble is stabilised with a thin shell, i.e. the shell is from negligible thickness up to about one tenth of the radius of the microbubble or nano bubble. Typically, a gas-filled microparticle has a nucleus-shell structure where the thickness of the shell or envelope may vary from a few nanometers to a few micrometers. Examples of gas-filled microparticles and of the preparation thereof are disclosed for instance in US patents U.S. Pat. No. 5,711,933 and U.S. Pat. No. 6,333,021. A variety of forms of microbubbles and nano bubbles are known in the art including the referenced soft and hard shell types, nano-sized and stabilised perfluorcarbon liquid drops which enlarge after ultrasound treatment only, thus acquiring their ultrasound contrast activity. In some embodiments a gas filled microparticle is included in a suspension in which the bubbles of gas are surrounded by a solid material envelope, an envelope of a lipid or an envelope of a natural or synthetic polymer. In some embodiments a microbubble, nano bubble or microparticle may have a specific type of electrostatic surface charge, having a specific chemical composition of the shell or consist of different types of gas such as e.g., air, nitrogen, oxygen, carbon dioxide, a noble gas, an alkane, an alkene, an alkine or a perfluoro-carbon-hydrogen or mixture of different type of gases. As an illustrative example, European patent application EP 0 921 807 describes the administration of gas-mixtures containing hydrogen gas may in liposomes, or gas-filled microparticles to patients. Administration of liquids in microbubbles or micro particles is for instance described in US patent application US 2003/0157023.

A microbubble generally includes a fluid and is edged/circumscribed by a thin envelope that involves a stabilised amphiphilic material. Once a respective microbubble is encompassed by body fluid of an organism, this amphiphilic material can be taken to be disposed at the gas to liquid interface. A microbubble may be provided in the form of a suspension of microbubbles in a liquid. Microbubble suspensions may be prepared by contacting powdered amphiphilic materials, e.g. freeze-dried preformed liposomes or freeze-dried or spray-dried phosphorlipids solutions, with air or other gas and then with an aqueous carrier, while agitating to generate a microbubble suspension which can then be administered, for example shortly after its preparation. Examples of aqueous suspension of gas microbubbles and preparations thereof are disclosed for instance in US patents U.S. Pat. No. 5,271,928 or U.S. Pat. No. 5,445,813.

As indicated above, in some embodiments of a method of detecting acute renal allograft rejection as described herein the probe includes a microbubble or a nanoparticle, to which a binding molecule is coupled. The probe is capable of specifically binding to T lymphocytes. In this regard the surface and/or the shell of a nanoparticle, microparticle or microbubble may include one or more moieties or molecules that have an affinity for a moiety or a molecule on the surface of T lymphocytes (supra). Having entered the blood stream of the subject's organism, the microbubble or nanoparticle thus gets immobilized on the surface of a T lymphocyte. In other words T lymphocytes become labelled with a contrast agent. Upon irradiation of graft tissue with ultrasound areas of such tissue where an increased number of T lymphocytes are found show a signal upon detection of sound waves, typically molecular imaging, i.e. sonography. The respective detection may in some embodiments be a Doppler ultrasound measurement.

In some embodiments of a method of detecting acute renal allograft rejection disclosed herein a time interval in the range from about a minute to about 20 minutes is allowed to pass before the graft of the subject is being exposed to ultrasound. This period of time may allow unbound freely circulating probes to be washed out of the graft and be cleared from the subject's blood pool by action of lungs and liver.

In some embodiments the signals obtained are compared to a threshold level. As an illustrative example, the intensity of echo signals obtained from an allograft may be assessed or evaluated and compared to the intensity of echo signals of a reference. A respective reference may be an average signal intensity of healthy kidneys obtained under the same or at least under comparable conditions. In embodiments where signals are quantified a threshold value may be used, i.e. the obtained signal strength may be compared to a threshold value. A respective threshold value may in some embodiments be a predetermined threshold value. In some embodiments the threshold value is based on the amount of cells having CD3, CD4 and/or CD8 in a control sample. In some embodiments the threshold value is based on the amount of cells having CD154, CTLA-4 and/or CD62L in a control sample. Such a control sample may be a sample of, include or essentially consist of the corresponding tissue as the tissue of the subject. A control sample may for example be a kidney of a healthy individual of comparable age, possibly of comparable history, as the subject.

In some embodiments a threshold value is based on a control or reference value obtained concomitantly with the detection of T lymphocytes in the subject's allograft. In some embodiments a respective control or reference value is determined at a different point in time, for example at a point in time earlier than the measurement of the sample from the subject is carried out. It is understood that the terms control and reference may in some embodiments be a range of values.

Population studies may also be used to select a threshold value. Receiver Operating Characteristic (“ROC”) arose from the field of signal detection theory developed during World War II for the analysis of radar images, and ROC analysis is often used to select a threshold able to best distinguish a diseased subpopulation from a nondiseased subpopulation. A false positive in this case occurs when a person tests positive, but actually does not have the disease. A false negative, on the other hand, occurs when the person tests negative, suggesting the person is healthy, when it actually does have the disease. To draw a ROC curve, the true positive rate (TPR) and false positive rate (FPR) are determined as the decision threshold is varied continuously. Since TPR is equivalent with sensitivity and FPR is equal to 1—specificity, the ROC graph is sometimes called the sensitivity vs (1—specificity) plot. A perfect test will have an area under the ROC curve of 1.0; a random test will have an area of 0.5. A threshold is selected to provide an acceptable level of specificity and sensitivity.

In addition to threshold comparisons, other methods for correlating assay results to a patient classification (occurrence or nonoccurrence of disease, likelihood of an outcome, etc.) include decision trees, rule sets, Bayesian methods, and neural network methods. These methods can produce probability values representing the degree to which a subject belongs to one classification out of a plurality of classifications.

The comparison to a threshold value, which may be a predetermined threshold value, can be carried out manually, semi-automatically or in a fully automated manner. In some embodiments the comparison may be computer assisted. As an illustrative example the overall signal intensity and/or the average signal intensity obtained from signals of T lymphocytes in the kidney may be counted. A computer assisted comparison may employ values stored in a database as a reference for comparing an obtained value or a determined amount, for example via a computer implemented algorithm. Likewise, the comparison to a reference measurement may be carried out manually, semi-automatically or in a fully automated manner, including in a computer assisted manner. A computer assisted comparison may rely on the storage of data, for instance in connection with determining a threshold value, on the use of computer readable media. Suitable computer readable media may include volatile, e.g. RAM, and/or non-volatile, e.g. ROM and/or disk, memory, carrier waves and transmission media such as copper wire, coaxial cable, fibre optic media. Exemplary carrier waves may take the form of electrical, electromagnetic or optical signals conveying digital data streams along a local network or a publically accessible network such as the Internet.

The level of T lymphocytes in a kidney may be expressed in terms of cell numbers, e.g. the number of T cells that are positive for CD3, for CD4, CD8, CD154, CTLA-4 and/or for CD62L. The level of expression of e.g. CD3, CD4 and/or CD8 may also be expressed in terms of the total amount of CD3, CD4 and/or CD8 in a sample. A significant difference of the obtained values may for instance indicate a high amount of CD3 or of CD3 positive cells in the sample. The term “significant” is used to indicate that the level of decrease or increase is of statistical relevance, and typically means a deviation of a value relative to another value of about 2 fold or more, including 3 fold or more, such as at least about 5 to about 10 fold or even more.

A predetermined threshold value may in some embodiments be set on the basis of data collected from one or more healthy subjects known not to have undergone renal transplantation and not to suffer from a renal condition or disease. In some embodiments a certain percentile of such data may be used as a threshold value. The range of the values of a set of data obtained from such individuals can be divided into 100 equal parts, i.e. percentages of the range can be determined A percentile represents the value within the respective range below which a certain percent of the data fall, in other words the percentage of the values that are smaller than that value. For example the 95th percentile is the value below which 95 percent of the data are found. In some embodiments a level of CD3, CD4, CD8, CD154, CTLA-4 and/or CD62L may be regarded as decreased or low if it is below the 90^{th} percentile, below the 80^{th} percentile, below the 70^{th} percentile, below the 60^{th} percentile, below the 50^{th} percentile or below the 40^{th} percentile.

In some embodiments a plurality of measurements is carried over a period of time at certain time intervals, including at predetermined time intervals. Such an embodiment may be taken as a method of monitoring the number of CD3, CD4, CD8, CD154, CTLA-4 and/or CD62L cells in the subject's kidney. The average value may be determined and the standard deviation calculated for each given time point. A value determined in the subject's tissue falling outside of the mean plus 1 standard deviation may be indicative of an occurrence of acute allograft rejection.

The present invention also provides a method of treating a subject. The method includes administering an immunosuppressive agent to the subject that/who has received a renal allograft. The method further includes administering to the subject a probe, which can specifically bind to T lymphocytes. The probe can be detected by ultrasound (supra). An allotransplanted kidney of the subject is exposed to ultrasound and the level of T lymphocytes in the kidney is detected. According to the level of T lymphocytes detected the amount or the nature of the immunosuppressive agent administered is being maintained, or adapted and/or changed. If an increased amount of T lymphocytes in the transplanted kidney is detected the immunosuppressive agent is exchanged or its dosage is being adapted.

A suitable immunosuppressive agent may be a glucocorticoid, an antimetabolite, a calcineurin inhibitor, a mTor inhibitor, an α4/β7 integrin modulator or a combination of any two or more such agents. Examples of a glucocorticoid include, but are not limited to, cortisone, hydrocortisone (cortisol), prednisone, aldosterone, prednisolone, methylprednisolone, dexamethasone, beclometasone, betamethasone, triamcinolone, fludrocortisone acetate and deoxycorticosterone acetate. Examples of an antimetabolite include, but are not limited to, an inosine monophosphate dehydrogenase inhibitor such as mycophenolate mofetil, a purine analogue, e.g. azathioprine or mercaptopurine, a pyrimidine analogue, a folic acid analogue, e.g. methotrexate, a serine palmitoyltransferase inhibitor such as myriocin and a protein synthesis inhibitor. Two illustrative examples of a calcineurin inhibitor are tacrolimus and cyclosporine. An example of a mTor inhibitor is the bacterial macrolide lactone sirolimus. An example of an α4/β7 integrin modulator is fingolimod. A further illustrative example of a suitable immunosuppressive agent is anti-thymocyte globulin (ATG), which at least essentially consists of horse or rabbit-derived antibodies against human T cells. Yet further examples of suitable immunosuppressive agents are the monoclonal immunoglobulins Atorolimumab, Cedelizumab, Fontolizumab, Maslimomab, Morolimumab, Pexelizumab, Reslizumab, Rovelizumab, Siplizumab, Talizumab, Telimomab aritox, Vapaliximab and Vepalimomab. Additional examples of suitable immunoglobulins have been named above. Suitable illustrative immunosuppressive agents are further CTLA-4 (available e.g. as Abatacept or Belatacept), a TNF inhibitor such as Etanercept or Pegsunercept, a VEGF inhibitor such as Aflibercept, an interleukin 1 inhibitor such as Rilonacept, as well as Alefacept.

Suitable routes of administration of compounds/agents used in the context of the present invention may, for example, include depot, oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into a solid tumor, often in a depot or sustained release formulation. Furthermore, one may administer the drug in a targeted drug delivery system, for example, in a liposome coated with a blood-cell specific antibody. The liposomes will be targeted to and taken up selectively by the respective cells.

Pharmaceutical compositions that include the compounds of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers including excipients and auxiliaries that facilitate processing of the active compounds into preparations that can be used pharmaceutically. Proper formulation is dependent upon the route of administration chosen.

For injection, the agents of the invention may be formulated in aqueous solutions, for instance in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated.

Pharmaceutical preparations for oral use can be obtained by adding a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethyl-cellulose, and/or polyvinylpyrrolidone (PVP).

If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoro-methane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a co-solvent system including benzyl alcohol, a non-polar surfactant, a water-miscible organic polymer, and an aqueous phase. The co-solvent system may be the VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD: D5W) consists of VPD diluted 1:1 with a 5% dextrose in water solution.

This co-solvent system dissolves hydrophobic compounds well, and itself produces low toxicity upon systemic administration. Naturally, the proportions of a co-solvent system may be varied considerably without destroying its solubility and toxicity characteristics.

Furthermore, the identity of the co-solvent components may be varied: for example, other low-toxicity non-polar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various types of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

A pharmaceutical composition also may include suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

For any compound used in the methods of the invention, the therapeutically effective dose can be estimated initially from cell culture assays. For example, a dose can be formulated in animal models to achieve a circulating concentration range that includes the IC₅₀ as determined in cell culture (i.e., the concentration of the test compound which achieves a half-maximal inhibition of the desired activity). Such information can be used to more accurately determine useful doses in humans.

Toxicity and therapeutic efficacy of the compounds described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio between LD₅₀ and ED₅₀. It may be desired to use compounds that exhibit high therapeutic indices. The data obtained from these cell culture assays and animal studies can be used in formulating a range of dosage for use in humans. The dosage of such compounds lies typically within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g., the concentration necessary to achieve 50-90% inhibition of the kinase. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen that maintains plasma levels above the MEC for 10-90% of the time, for example from about 30 to about 90%, such as from about 50 to about 90%.

In cases of local administration or selective uptake, the effective local concentration of the drug may not be related to plasma concentration. The amount of composition administered will, of course, be dependent on the subject being treated, on the subject's weight, the severity of the affliction, the manner of administration and the judgment of the prescribing physician.

A suitable composition may, if desired, be presented in a pack or dispenser device which may contain one or more unit dosage forms containing the active ingredient. The pack may for instance include metal or plastic foil, such as a blister pack. The pack or dispenser device may be accompanied by instructions for administration. The pack or dispenser may also be accompanied with a notice associated with the container in a form prescribed by a governmental agency regulating the manufacture, use, or sale of pharmaceuticals, which notice is reflective of approval by the agency of the form of the compound for human or veterinary administration. Such notice, for example, may be the labeling approved by the U. S. Food and Drug Administration or other government agency for prescription drugs, or the approved product insert.

A subject may be first subjected to prior screening to determine whether the planned treatment would be suitable. For example, such a screening may be based on the patient history, previous use of immunosuppressant, Expanded Disability Status Scale (EDSS), MRI imaging studies, pre-infusion checklist for continuously worsening neurological symptoms, and other criteria commonly used.

The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the appending claims. In addition, where features or aspects of the invention are described in terms of Markush groups, those skilled in the art will recognize that the invention is also thereby described in terms of any individual member or subgroup of members of the Markush group.

In order that the invention may be readily understood and put into practical effect, particular embodiments will now be described by way of the following non-limiting examples

### Examples

### Labeling of Microbubbles

Microbubbles (Bracco, Konstanz, Germany) containing Streptavidin were labeled with biotinylated anti-CD3-antibodies (Abcam, Cambridge, United Kingdom) by incubation for 15 min at room temperature.

### Image Acquisition

Image acquisition using a high resolution HDI 5000 Ultrasound system (Philips) was performed 1-4 days after transplantation under general anesthesia delivered through a nose mask while heart rate and body temperature were maintained. 15 min after tail vein injection of 30×10⁶ human T lymphocytes a base line image of both kidneys was recorded followed by the application of 100 μl anti-CD3 labeled microbubbles. After another 2 min a contrast enhanced image was acquired and animals were immediately sacrificed and kidneys were isolated and prepared for histological analysis.

### Detection of Acute Renal Allograft Rejection Based on Detecting Labeled T-Lymphocytes

### Abstract of this Section

Provided is also a method of detecting acute renal allograft rejection in a subject based on using identifiably labeled T-lymphocytes. The labeled lymphocytes may be detected by means of any suitable technique. In the following, small animal positron emission tomography (PET) is employed to this effect. T-lymphocytes labeled with ¹⁸F-fluorodeoxyglucose (¹⁸F-FDG) have been used in the embodiment described in more detail. This method can be taken to be an image-based diagnosis of acute allogeneic renal transplant rejection (AR). To illustrate the method a rat model is used as an example.

### Examples

1 h and 2 h after tail vein injection of 30×10⁶ ex vivo ¹⁸F-FDG labeled human T-cells into male 10 weeks old uni-nephrectomized, allogeneically transplanted rats (Lewis-Brown Norway (LBN) to Lewis, aTX), whole body radioactivity distribution was assessed in vivo by small animal PET (post operative day 4), and percentage of injected dose (% ID) as a parameter of T-cell infiltration was assessed and compared between graft and native kidney. In vivo results were confirmed by autoradiography and staining of human CD3 after post mortem dissection. Syngeneically (sTX) transplanted rats (LBN to LBN), rats with ischemia/reperfusion injury (IRI, 45 min warm ischemia), and rats subjected to acute cyclosporine A toxicity (CSA) (50 mg/kg for 2 days i.p.) served as controls.

### Results

Accumulation of labeled cells was significantly elevated in allografts with AR (1.07±0.28% ID) compared to native control kidneys (0.49±0.18% ID) (P<0.0001). No differences were found between native controls, sTX, CSA and kidneys with IRI. In vivo uptake of ¹⁸F-FDG cells measured in the PET scanner correlated with results obtained by autoradiography, histological evaluation and PCR.

### Conclusion

We propose graft PET imaging using ¹⁸F-FDG labeled T-cells as a new option to detect rat renal AR with very low dose of ¹⁸F-FDG in a non-invasive, fast and very specific manner in rats.

### INTRODUCTION

As already explained in the preceding section, at present, diagnosis of acute transplant rejection (AR) in humans relies on graft biopsy upon renal allograft dysfunction(1, 2). However, more than 50% of the rejection episodes are subclinical without acute impairment of renal function and, interestingly, with a histological severity score which was found to be comparable to that of patients with a decrease in glomerular filtration rate during AR(3). Therefore, one may take protocol biopsies at a defined time-course after transplantation, irrespective of the status of graft function, to diagnose AR(2). Biopsy is an invasive procedure which is cumbersome to the patient, carries the risk of graft injury, and is not feasible in recipients taking anticoagulant medication(4, 5). In addition, the sampling site is small and AR may be missed, i.e., when rejection is focal or patchy. Thus, in diagnostics, entirely image-based methods visualizing the whole organ would be superior. Nuclear imaging approaches including single photon emission computer tomography (SPECT) and positron emission tomography (PET) have the advantages of high intrinsic sensitivity, excellent tissue penetration, and a wide range of clinically available molecular imaging probes(6). Recently, we have established ¹⁸F-FDG-PET as an entirely image-based method to assess and to monitor acute renal rejection(7, 8). However, potential drawbacks in applying ¹⁸F-FDG-PET to the clinical setting should be noted. First, urinary excretion of the tracer necessitates late acquisitions to reduce the amount of ¹⁸F-FDG in the urine. Further, the renal pelvis should be carefully excluded from the measurements. Second, ¹⁸F-FDG-uptake is not disease-specific. Thus, a more specific method is desirable.

AR results from interactions between the recipient's immune system and the foreign antigens serving as a target. T-lymphocytes (CD4⁺ and CD8⁺) are central and specific to the AR process whereas B cells and the congenital immune system, e.g., complement, monocytes/macrophages, neutrophils, and dendritic cells, participate(9, 10). After recognition of donor-derived antigens, T-cells migrate into the transplant and infiltrate the interstitial space (11). Since recruitment and activation of inflammatory cells, in particular lymphocytes, play decisive roles in AR, efforts have already been made in order to image infiltration by means of radiolabeled leukocytes (6). Because infiltration of leukocytes, especially T-lymphocytes in allografts, appears before physiologic or mechanical manifestations of organ dysfunction is apparent, nuclear imaging employing lymphocytes might be a promising tool for sensitive and early detection of rejection. However, PET with ¹⁸F-FDG labeled T-lymphocytes for diagnostics of AR has never been tested before. Thus, we applied PET with ¹⁸F-FDG labeled human T-lymphocytes in an established rat renal transplant model(8). ¹⁸F-FDG-uptake of the renal parenchyma was assessed on postoperative day 4 (POD4) in uni-nephrectomized, allogeneically kidney-transplanted animals (aTX) with, and additionally in native controls (CTR) and syngeneically transplanted (sTX) animals without rejection or impairment of renal function. Because acute cyclosporine A nephrotoxicity (CSA) and acute tubular necrosis (ATN) caused by ischemia/reperfusion injury (IRI) are important differential diagnosis of AR, we have included these two additional groups into the study.

The method disclosed in this section is a method of detecting acute renal allograft rejection in a subject. The method includes administering T lymphocytes to the subject. The T lymphocytes are in some embodiments T lymphocytes of the same subject. The T lymphocytes may for instance have been obtained from the subject at an earlier point in time and subsequently labelled. In some embodiments the T lymphocytes are lymphocytes of a different subject of the same species. In some embodiments the T lymphocytes are lymphocytes of a subject of a different species.

Coupled to the T lymphocytes or included in the T lymphocytes, which are being administered to the subject, is a detectable label. The label may be a radioactive label such as a moiety of a radioactive metabolite. In some embodiments the label is detectable by applying positron emission tomography. Where positron emission tomography is to be used for detection, the positron-emitting radioactive isotope fluorine-18 may be a suitable moiety included in a T lymphocyte. As an illustrative example, a commonly used compound in this regard is ¹⁸F-fluorodeoxyglucose.

The method may further include waiting for a period of time. This period of time may be a predetermined period of time. The time interval chosen for waiting may be from about 2 minutes to about 5 hours. In some embodiments the time interval is from about 10 minutes to about 3 hours. In some embodiments the time interval is about 1 hour. In some embodiments the time interval is about 2 hours.

The method also includes detecting the label in an allotransplanted kidney of the subject. Thereby the method includes detecting the level of T lymphocytes in the kidney. In typical embodiments the level of T lymphocytes in the kidney is being detected by detecting signals generated by the label. In some embodiments the detection includes applying an imaging technique. In one embodiment the imaging technique is positron emission tomography. An elevated level of T lymphocytes in the kidney indicates acute renal allograft rejection.

### Examples

### Materials and Methods

### Animal Models

Surgical and imaging experiments were approved by a governmental-committee on animal welfare and were performed in accordance with national animal protection guidelines. Male Lewis-Brown-Norway (LBN) and Lewis (LEW) rats (200-270 g body weight (BW), Charles River, Sulzfeld, Germany) with free access to standard rat chow (Altromin, Lage, Germany) and tap water were used. Surgeries were performed under anaesthesia with ketamine 100 mg/kg body weight intra peritoneal (i.p.) and xylazine 5 mg/kg BW i.p. (Xylazin, Ketamin, CEVA Tiergesundheit, Düsseldorf, Germany). Further doses of ketamine were injected as needed.

Transplantation was simultaneously performed by two investigators as published before(8, 12, 13). In short, the left kidney including ureter, renal artery, a piece of aorta and renal vein were transferred into the recipient. Kidneys from age- and weight-matched LBN were transplanted into LEW (aTX). Transplantations were performed immediately after left nephrectomy of the recipient. While the total operation time of the recipient did not exceed 90 min, the ischemia time of the graft was always shorter than 40 min. Grafts were studied on POD4 after transplantation. The chosen aTX model leads to histological and functional changes typical for AR(8, 12, 13). We have previously reported functional and histological signs of AR occurring clearly on POD4(8, 12, 13). As in this AR model without immunosuppression graft necrosis starts only a few days beyond POD4 we decided to investigate kidneys on POD4. Syngeneically transplanted rats (LBN to LBN, sTX) without AR served as controls. Because acute tubular necrosis (ATN) and acute cyclosporine toxicity (CSA) are common differential diagnoses of AR, these groups have been also included. ATN (induced by IRI) and acute CSA toxicity were induced as published before(8, 14). For IRI, the left renal artery was dissected as in TX-groups and ligated for 45 min using a microvascular clamp. After clamp release, the returning of original surface color of the kidneys was confirmed visually. For acute CSA-induced nephrotoxicity, rats received 50 mg/kg CSA (Sandimmun, Novartis, Nürnberg, Germany) i.p. for two days.

### T-Cell Isolation and Labeling

¹⁸F-FDG was produced in a clinical routine setup on site using an RDS 111 cyclotron (CTI, Knoxville, TE, USA). T-lymphocytes were isolated from human buffycoats (containing mainly white as well as red blood cells and platelets) purchased at the DRK Münster, Germany by negative antibody selection using the RosetteSep® method according to the manufacturer's protocol (Stemcell, Köln, Germany). After counting cell numbers in a Neubauer chamber, cells were adjusted to the desired amount, incubated with 80 to 120 MBq ¹⁸F-FDG for 30 min, washed twice with phosphate buffer, and finally resuspended in 300 μl physiological saline for further experiments.

For analysis of labeling efficiency 5×10⁶ T-cells were either incubated for 30 min with 7 MBq ¹⁸F-FDG in physiological saline or a K⁺ enriched solution (Sterofundin, Braun, Melsungen, Germany) in the presence or absence of 10 I.U. insulin. After centrifugation, radioactivity was measured insupernatant as well as pelleted cells by gamma counting and labeling efficiency was calculated as the ratio between radioactivity in cells and overall radioactivity.

For analysis of labeling stability 5×10⁶ T-cells labeled with 7 MBq ¹⁸F-FDG as described before were incubated in blood plasma for 10, 30, 60 and 120 min respectively. After incubation samples were centrifuged, and radioactivity was measured of supernatant as well as of pelleted cells by gamma counting. Labeling stability was calculated as percentage share of radioactivity in cells/supernatant based on total radioactivity.

### Image Acquisition—PET

Image acquisition of non-fasting subjects was performed as described before(7, 8). In short, T-cell infiltration was calculated from a whole body acquisition of 20 min length 1 and 2 hours after injection of 30×10⁶ ex vivo ¹⁸F-FDG labeled human T-cells (1-2 MBq ¹⁸F-FDG) in 300 μl 0.9% NaCl solution into a tail vein. Thereafter, the catheter was purged with additional 700 μl 0.9% NaCl solution. Rats remained in a restrainer under anaesthesia until start of the scan. During acquisition, rats were further anaesthetized with oxygen/isoflurane inhalation (2% isoflurane, 0.7 l/min oxygen) and body temperature was maintained at physiological values by a heating pad. PET scans were performed with a high-resolution multi wire chamber-based animal PET camera (quadHIDAC, Oxford Positron Systems Ltd, Oxford, UK)(15).

### Analysis of PET Images and Quantitative Evaluation

A renal parenchyma volume of interest (VOI) was manually traced around the kidneys on reconstructed coronal images. Mean ¹⁸F-FDG-T-cell activity of the renal parenchyma was calculated by the ratio of total counts and volume.

### Autoradiography

To validate the data obtained by PET, animals were sacrificed and kidneys were excised immediately after PET scanning. High-resolution autoradiography (μ-imager, Biospace Measures, Paris, France) was performed as published before(8). In short, kidneys were snap-frozen, sliced into 10 μm thick cryosections and the radioactivity was measured in a mid-coronary renal slice for 3 h.

### Histology

Portions of kidneys were snap-frozen and fixed in 4% formaldehyde in PBS. Histological changes (glomerulitis, tubulitis, endothelialitis, and infiltration) were examined by light microscopy in paraffin-embedded tissue with PAS and HE staining. For the evaluation, only cortex was chosen as the extent of medullary inflammation does not reflect the degree of AR(16).

### Immunohistochemistry

After fixation in 4% formaldehyde in PBS and embedding in paraffin, 3-μm-thick slices of kidneys were deparaffinized with Clear Rite and rehydrated in descending ethanol series. Sections were then blocked with BSA 10% and immunostained using the ABC method with antibodies against human CD3, subunit epsilon (Abcam, Cambridge, United Kingdom) and alkaline phosphatase with antibodies against CD3 epsilon (Thermoscientific, Bonn, Germany). After counterstaining with Haemalaun, images were acquired using an Axio Zeiss microscope (Axiovert 100, Carl Zeiss AG, Oberkochen, Germany) equipped with a digital camera (AxiocamMRc, Carl Zeiss AG) using the AxionVisonLE Release 4.7.1 software (Carl Zeiss AG). Control stainings included images without primary antibody. The number of CD3 positive cells was quantified in 10 field of views (FOV, 350×250 μm each) using ImageJPixFRET software (17) (downloadable at http://rsb.info.nih.gov/ij/).

### Flow Cytometry

T-lymphocyte purity of isolated cells was verified by staining with CD4⁺-APC, CD8⁺-PE, CD3⁺-FITC and CD45⁺-PerCP-Cy5.5 (BD Biosciences, Heidelberg, Germany) and subsequent FACS analysis as described previously (18).

### Real Time PCR

Expression profiles of selected marker genes for infiltrating cells were validated by real time PCR which was performed using SYBR Green PCR Master Mix or TaqMan Universal PCR Master Mix on an ABI PRISM 7700 Sequence Detection System. Specific primer pairs were used as listed in Table 1. All instruments and reagents were purchased by Applied Biosystems (Darmstadt, Germany). Relative gene expression values were evaluated with the 2^{-ΔΔCt} method using Gapdh as housekeeping gene.

### Statistics

Data was compared by ANOVA with a Scheffé multiple comparisons test. Data is presented as mean values±SEM (n=number of rats, samples, or experiments). Significance was inferred at the P<0.05 level.

### Results

### Flow Cytometry and Labeling of Isolated T-Lymphocytes

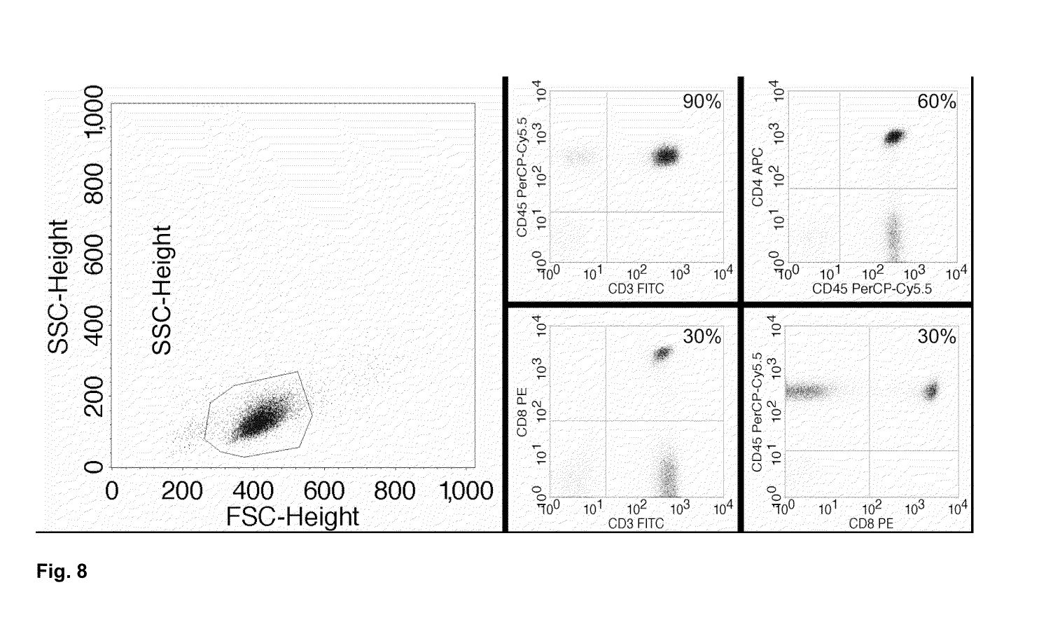

Flow cytometry analysis with CD45 as a general marker for leukocytes and CD3 as a specific marker for T-cells revealed a mean T-lymphocyte purity of approximately 90% (n=3). FIG. 8 shows a representative experiment, where about 60% of isolated cells belong to the subpopulation of CD4⁺ T-helper cells, whereas about 30% are part of the group of CD8⁺ cytotoxic T-effector cells (CD4⁺/CD8⁺ ratio: 1,9).

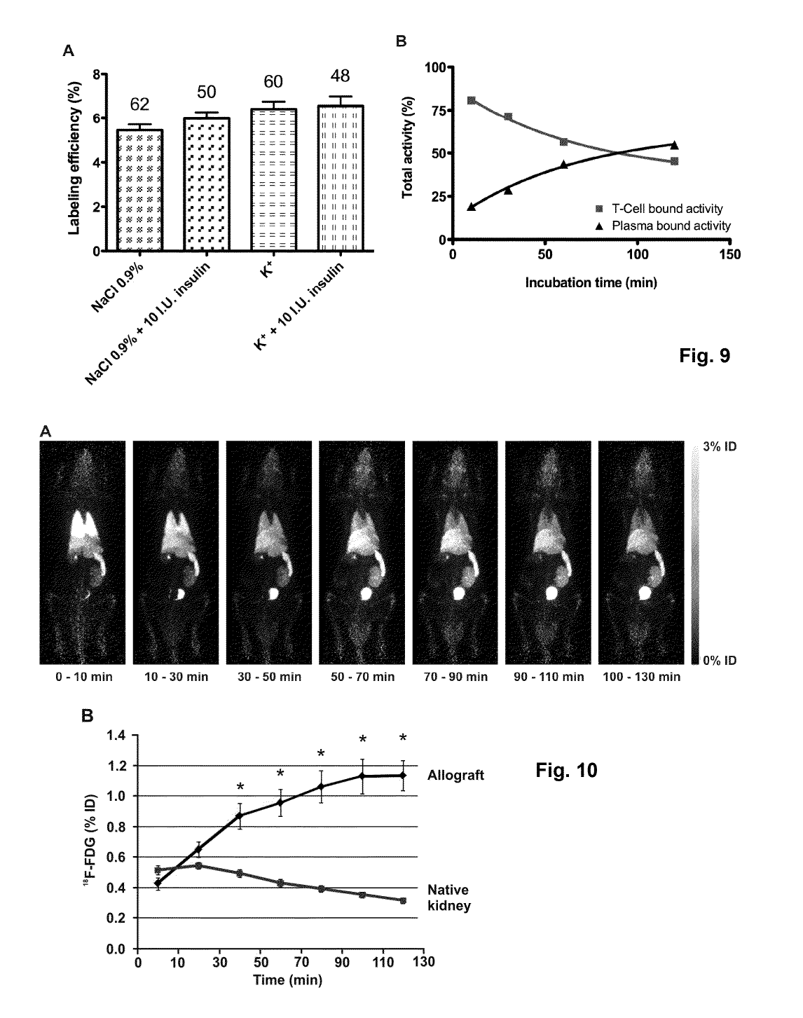

The mean labeling efficiency of T-lymphocytes with ¹⁸F-FDG using physiological saline was 5.4±2.1%. In order to elevate ¹⁸F-FDG labeling efficiency by means of stimulating insulin-dependent glucose transporters e.g. GLUT4, T-cells were co-incubated with insulin and potassium. However, neither the addition of insulin (6.0±2.0%) nor potassium (6.4±2.6%), nor the combination of insulin and potassium (6.6±3.0%) significantly increased the ¹⁸F-FDG uptake in T-lymphocytes (FIG. 9A).

Labeling stability slowly decreased in vitro over time. Retention of the radioncuclide in the cells declined from 80.7±0.64% after 10 min over 71.25±0.46% after 30 min to 56.42±1.14% after 60 min and reached 45.2±0.97% after 120 min (FIG. 9B).

### Analysis of PET Images and Quantitative Evaluation

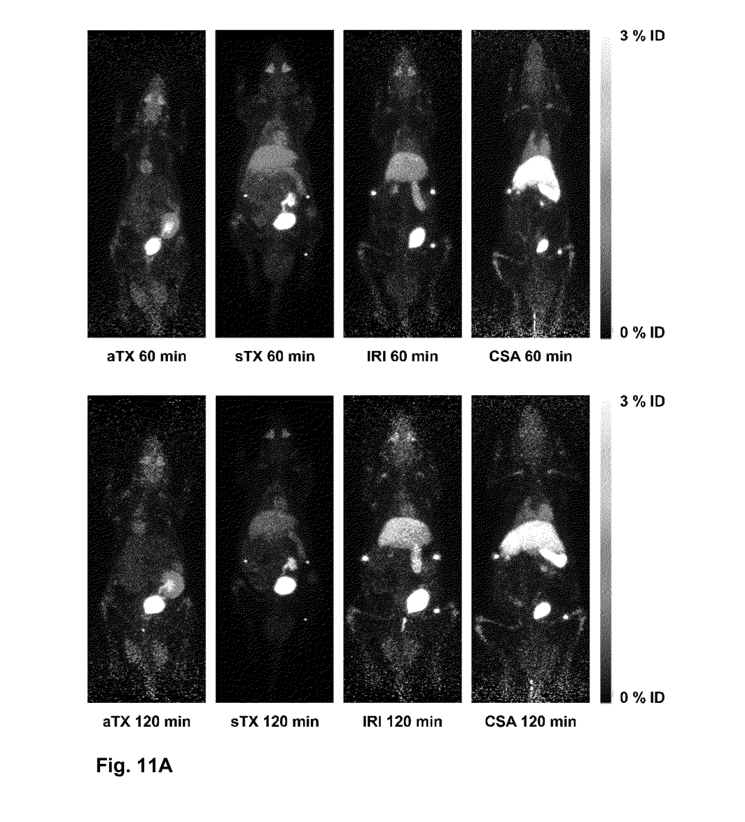

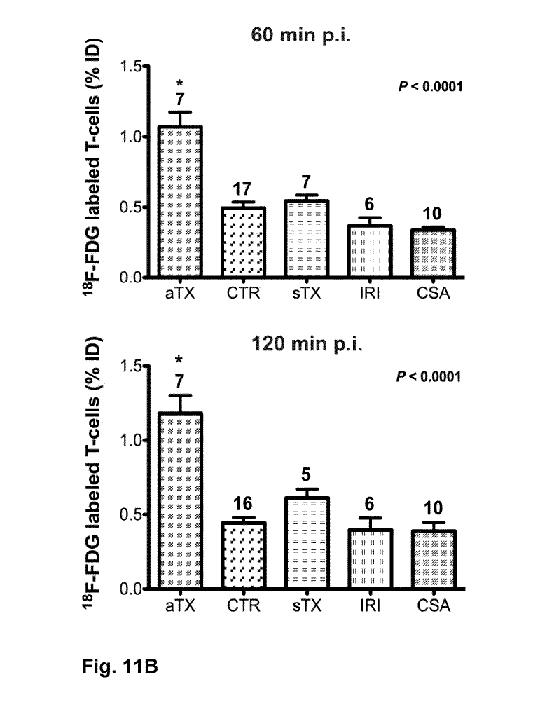

In allografts undergoing AR (POD4), we detected a clearly elevated T-lymphocyte signal already 1 h p.i., which had only slightly increased further after 2 h (FIGS. 10 and 11). Quantification of ¹⁸F-FDG labeled T-cell accumulation as percentage of injected dose (% ID) within the parenchyma of the investigated kidneys revealed a significant increase in allografts only (aTX: 1.07±0.28% ID after 1 h, n=7, and 1.18±0.32% ID after 2 h, n=7, P<0.0001 vs. all other controls), when compared to native control kidneys (0.49±0.18% ID, n=17, and 0.44±0.15% ID, n=16) as well as sTX (0.55±0.11% ID, n=7, and 0.61±0.14% ID, n=5), CSA (0.34±0.07% ID, n=10, and 0.39±0.18% ID, n=10) and kidneys with IRI (0.37±0.14% ID, n=6, and 0.40±0.20% ID, n=6) (FIG. 11B).

### Autoradiography

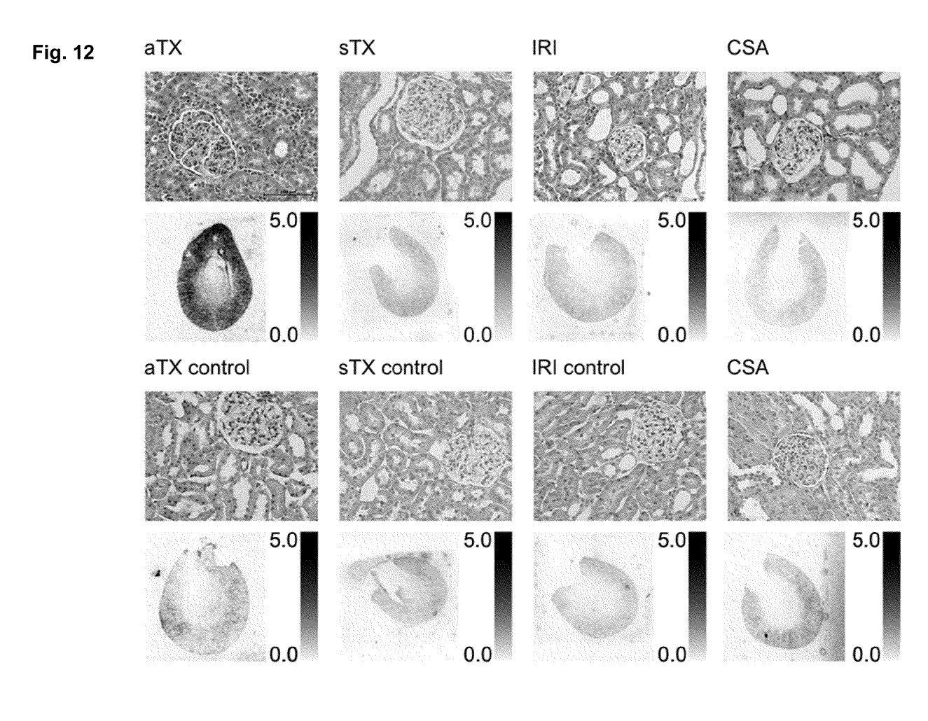

Assessment of inflamed tissue by autoradiography confirmed that accumulation of ¹⁸F-FDG labeled cells correlates to the degree of infiltration. Therefore, we have chosen autoradiography as a reference method to validate PET results with ¹⁸F-FDG labeled T-lymphocytes. As described previously POD4 was chosen since accumulation of radiolabeled T-cells reached significance at that time, while graft integrity was still maintained. On POD4 mainly the renal cortex but also the medulla of aTX kidneys demonstrated a significant accumulation of radiolabeled cells (FIG. 12). Allografts exhibited a nearly 4-fold increased tracer uptake, when compared to native control kidneys, isografts, kidneys with IRI and kidneys with acute CSA toxicity (Table 2).

### Histology

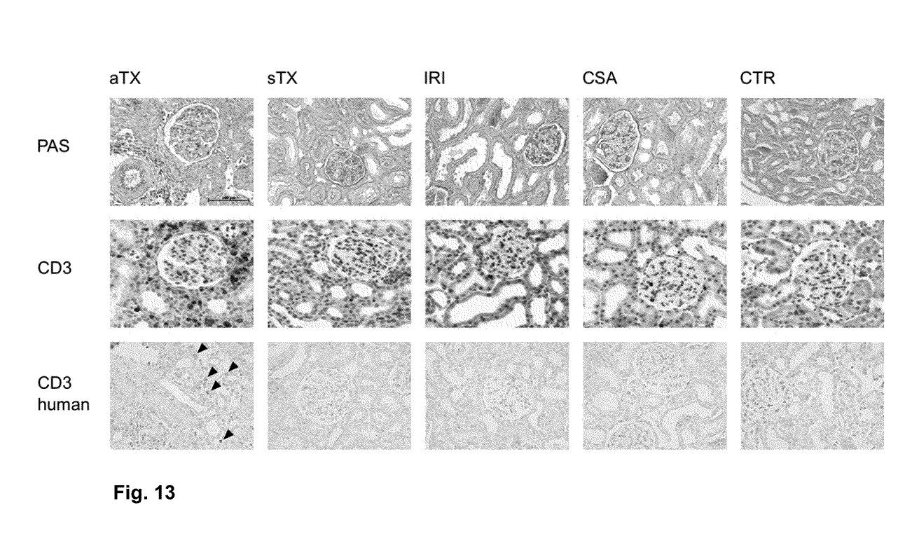

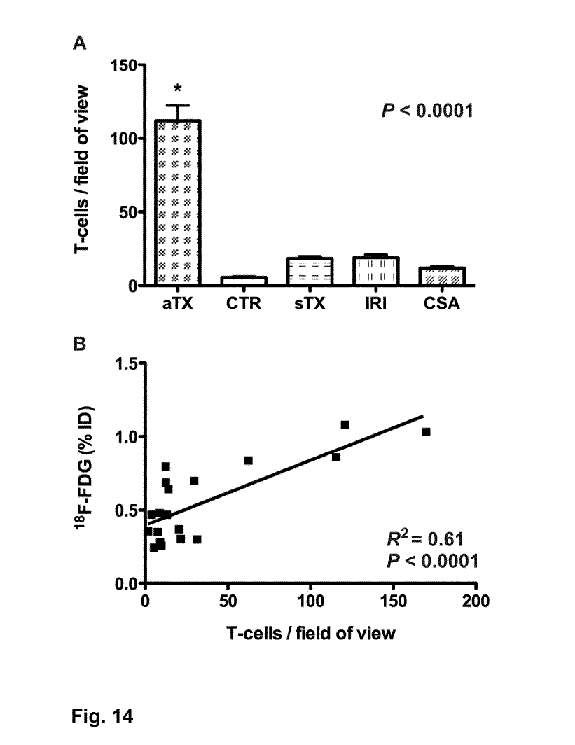

To estimate renal damage and infiltration for validation of ¹⁸F-FDG data we evaluated renal histology and quantified the number of CD3 positive infiltrating T-lymphocytes. In allografts, we found distinct signs of acute rejection (marked glomerulitis, tubulitis and endothelialitis) as well as a highly significant infiltration with CD3 positive T-cells in all grafts on POD4 (FIG. 13, 14A). As demonstrated, histological signs of rejection or significant infiltration were absent in controls (FIG. 13, 14A). However, renal damage according to the induced injury was present, e.g., mild tubulitis or detachment of cells into the tubular lumen (ATN) or hyaline arteriolar thickening (CSA). To validate our data, transferred T-lymphocytes were stained with a human specific antibody against the CD3 epsilon subunit. Although all kidneys were perfused for further histological analysis, at least some CD3 positive cells were found in the cortex of renal allografts undergoing acute rejection (aTX), whereas more or less no human cells could be documented in controls (sTX, CSA, ATN) (FIG. 13)

### Correlation of PET-Data and Histology

To verify our hypothesis that graft infiltration with radiolabeled T-cells is directly related to the degree of inflammation in AR we correlated the number of CD3 positive cells/FOV of each group to their according % ID. This correlation was found significant (R²=0.61) (FIG. 14B).

### Real Time (RT) PCR Analysis

We used RT-PCR analysis (n=5/group) to confirm and characterize inflammatory cell pattern in aTX. Analysis of aTX graft revealed distinct up-regulation of CD3 and CD8b on POD4 (Table 3). Up-regulation of CD3 and CD8b was absent in isografts (sTX), IRI and in CSA confirming the absence of AR or T-cell accumulation. Notably, mRNA expression of CD3 as well as CD8b significantly correlated with accumulation of ¹⁸F-FDG labeled T-cells (R²=0.41 and R²=0.40).

### Discussion

Episodes of AR are characterized by a distinct inflammation pattern(19), where leukocytes, mainly activated T-lymphocytes, are recruited into the transplant(20). The updated Banff classification categorizes infiltration and finally scores renal transplant rejection(1, 21, 22). At present, core needle biopsy is the gold standard in the definite diagnosis of AR. However, as an invasive method it bares the risk of severe graft injury. Moreover, it is not feasible in patients receiving anticoagulation therapy and might present false negative results, if AR is focal or patchy. Since activated leukocytes highly accumulate ¹⁸F-FDG, which can be assessed by PET, we recently established ¹⁸F-FDG PET for non-invasive detection and monitoring of renal allograft rejection(8). Moreover, ¹⁸F-FDG PET can be applied for the early evaluation of immunosuppressive treatment response and might assist in the differential diagnosis of AR, ATN, and acute CSA toxicity(7).

Nevertheless, potential drawbacks of the tracer ¹⁸F-FDG on the one hand are urinary excretion of ¹⁸F-FDG and drainage into the renal pelvis. This aggravates the assessment of parenchymal glucose metabolism and might induce false positive ¹⁸F-FDG signaling. Moreover, it necessitates late acquisitions to reduce the amount of urinary ¹⁸F-FDG. On the other hand, assessment of ¹⁸F-FDG uptake represents unspecific metabolic activity. Thus, especially graft infection or potentially also lymphoma might generate a similar ¹⁸F-FDG accumulation pattern(23). Although clinical symptoms as well as additional serological and image-based methods can assist in the differential diagnosis, a more specific tracer would be desirable. Since recruitment of T-lymphocytes does not play a leading role in the development of infection, this diagnosis most likely can be ruled out using PET with radiolabeled T-cells. Moreover, using ¹⁸F-FDG PET for detection of AR, PET assessment was applied 3 h after injection for several reasons such as ¹⁸F-FDG drainage or elimination of free ¹⁸F-FDG. Although this is earlier than diagnostics by histologic evaluation in most cases, an easy and fast translation into the clinics might be challenging.

In this study, we present PET with ex vivo ¹⁸F-FDG labeled human T-lymphocytes as a novel, highly specific diagnostic tool to assess AR occurring in an allogeneic rat renal transplantation model. Infiltration of radiolabeled T-lymphocytes in all used renal injury and transplant models was quantitatively evaluated from PET images and calculated as % ID. Allografts developing AR exhibit a significant elevation of % ID on POD4, when compared to isografts. POD4 was chosen, because signs of AR clearly occur on POD4(8), whereas graft perfusion and function is still preserved. Consistently, injured kidneys with common differential diagnoses of AR, namely ATN and acute CSA toxicity, did not show any accumulation of radiolabeled T-cells. Using ¹⁸F-FDG labeled leukocytes, Toso et al. tried to assess AR of pancreatic islet transplants in the liver. Their approach remained unsuccessful due to the well-known free ¹⁸F-FDG-uptake in the liver (background activity) (24).

In order to elevate ¹⁸F-FDG-labeling efficiency T-cells were co-incubated with insulin and K. However, neither the addition of insulin nor K⁺, nor the combination of insulin and K⁺ significantly increased the ¹⁸F-FDG uptake in T-cells (FIG. 9A). These results are concordant with those in the literature because T-lymphocytes mainly express the insulin-independent GLUT1 and GLUT3 (25) and do not respond to insulin with an up-regulation of glucose transporters(26). Interestingly, Botti et al. were able to demonstrate much higher labeling efficiencies. However, they used lower amounts of radioactivity for cell incubation resulting in a better ratio of radioactivity to cell number in their experiments (27).

To validate our PET data, we correlated the entirely imaged-based measurements of T-cell accumulation to results from reference methods, e.g. histological quantification of infiltration (leukocytes, CD3 positive T-lymphocytes) (FIG. 13) as well as mRNA expression of T-cell markers within the kidneys (Table 3). We found a significant correlation between enhanced accumulation of radiolabeled T-cells and histological infiltration (FIG. 14A, B). In contrast, native controls showed a very low ¹⁸F-FDG uptake only, which also correlated with the histological findings. In addition, syngeneically transplanted grafts, as well as kidneys with ATN and acute CSA toxicity, only exhibited marginal histological signs of infiltration, again correlating well with the ¹⁸F-FDG-uptake. These results were supported by real time PCR analysis of marker genes. As commonly observed during AR(9) we detected significant mRNA up-regulations of activated T-lymphocyte surface antigens such as CD3 and CD8b in renal allografts on POD4. This up-regulations were absent in sTX, ATN and CSA (Table 3). Notably, the expression of CD3 as well as CD8b significantly correlated with accumulation of ¹⁸F-FDG labeled T-lymphocytes in all measured groups.

As a third reference method, autoradiography revealed a significantly higher accumulation of radiolabeled cells in allogeneic transplants compared to native controls as well as kidneys with ATN, acute CSA toxicity or sTX (Table 2). In detail, allografts exhibited a nearly 4-fold increased activity when compared to control kidneys (native controls, sTX, IRI, and CSA). Taken together, these results indicate that PET with radiolabeled T-cells is not only able to diagnose, but also to quantify AR and thereby represent its histological degree of inflammation.

The use of radiolabeled cells bares several advantages in comparison to conventional ¹⁸F-FDG PET imaging. First, despite the very low amount of radioactivity contained within the radiolabeled T-cells, a very strong signal can be detected in renal allografts. This militates in favor of a potential higher sensitivity, whilst simultaneously reducing the patient's radiation dose to a minimum. Second, less urinary excretion of free ¹⁸F-FDG was documented, which potentially could lead to false positive results. In humans, PET imaging using ¹⁸F-FDG labeled leukocytes leads to less than 10% leakage of free ¹⁸F-FDG in the urinary system after 6 h(28). Our in vitro labeling stability studies revealed that the amount of free ¹⁸F-FDG slowly increases over time (FIG. 9B). This has also been shown by Botti et al. who assessed the liberation rate of FDG in T-cells(27). However, since urine microscopy after PET-scans did not reveal relevant amounts of T-cells (data not shown), the activity shown in the bladder accounts at least in part to free ¹⁸F-FDG.

Most likely, the activity was released by (destructed) T-cells. In cases of acute rejection, some of this FDG should be delivered by T-cells to the kidney and locally released. However, we assessed the bladder activity in all groups of rats and could not find differences between the groups (approximately 2-3% ID). Thus, liberation of activity equals in all groups leading us to the conclusion that activity is probably released from destructed cells, which have been affected e.g., by the immune response. This is supported by data from Pellegrino et al. who analyzed the form of FDG trapped in FDG-labeled white blood cells(29). They observed that the chemical form of ¹⁸F as analyzed by TLRC demonstrated that virtually all radioactivity inside the white blood cells was present as ¹⁸F-FDG-6P, a chemical form incapable of egression from the cell and minimally reconverted to ¹⁸F-FDG.

Third, using radiolabeled lymphocytes, PET can be applied as early as 1 h after injection thereby significantly accelerating the diagnostic procedure. The T-cell ¹⁸F-FDG signal of aTX subsequently increased after injection of T-cells reaching significant difference to the native kidney at 30-50 min after the injection. The time point 50-70 min was chosen because it combined a significant increase in ¹⁸F-FDG signal with a good visual delineation of the graft.

One might question the fact that xenogene cells were used for the detection of AR. However, this has been established before. Wang et al. performed in vivo PET imaging using human cells in a Parkinson's disease rat model(30) and Hay et al. used human ¹¹¹In labeled leukocytes to assess inflammatory lesions in rats(31). Moreover, imaging of total body T-cell distribution in our rats exhibited a distinct allocation with a primary emphasis on the reticulo-endothelial system and was comparable to the biodistribution of ¹⁸F-FDG labeled leukocytes in healthy humans.

Imaging using ex vivo radiolabeled leukocytes is a well established method that found its way into daily clinical routine particularly in the diagnosis of inflammatory and infectious disorders (e.g. scintigraphy using ¹¹¹In and ^{99m}Tc labeled leukocytes)(32-35). Since recruitment and activation of inflammatory cells, especially T-lymphocytes, play a pivotal role in AR, there have been efforts made to image graft infiltration by means of radiolabeled cells. SPECT-based imaging with ¹¹¹In and ^{99m}Tc labeled leukocytes has already been used to assess AR in a very small cohort of intestinal as well as kidney transplant recipients(36, 37). However, PET has the advantage of a quantitative analysis (assessment of % ID or standardized uptake value, SUV), which is useful in diagnosis of allograft rejection and for monitoring patients' responses to therapy. Thus, PET with ¹⁸F-FDG labeled autologous T-lymphocytes might be translated to humans in the near future.

### CONCLUSION

We present and validate an entirely imaging-based non-invasive method to assess AR using PET with ¹⁸F-FDG labeled T-lymphocytes. Requiring extremely low radioactive doses, this method is highly specific and can differentiate between AR and ATN. A clinical translation in order to investigate the kinetics of AR as well as the response to therapeutic intervention seems promising.

### ACKNOWLEDGEMENTS

This study was supported in part by the Interdisciplinary Centre for Clinical Research Munster, Germany (IZKF, Core Unit PIX), and the Deutsche Forschungsgemeinschaft (SFB 656 C7 & C6). The authors are grateful to Anne Kanzog, Christine Bätza, Ute Neugebauer, Rita Schröter, Wiebke Gottschlich, Sandra Schröer, Irmgard Hoppe and Roman Priebe for excellent technical assistance and to Daniel Burkert and Sven Fatum for producing radiotracers.

### REFERENCE LIST

### Second Section of the Document

(1) Racusen L C, Solez K, Colvin R B et al. The Banff 97 working classification of renal allograft pathology. Kidney Int. 1999; 55:713-723.
(2) Rush D. Protocol transplant biopsies: an underutilized tool in kidney transplantation. Clin J Am Soc Nephrol. 2006; 1:138-143.
(3) Morath C, Ritz E, Zeier M. Protocol biopsy: what is the rationale and what is the evidence? Nephrol Dial Transplant. 2003; 18:644-647.
(4) El-Mekresh M, Osman Y, Ali-El-Dein B, El-Diasty T, Ghoneim M A. Urological complications after living-donor renal transplantation. BJU Int. 2001; 87:295-306.
(5) Schwarz A, Hiss M, Gwinner W, Becker T, Haller H, Keberle M. Course and relevance of arteriovenous fistulas after renal transplant biopsies. Am J Transplant. 2008; 8:826-831.
(6) Grabner A, Schnockel U, Kentrup D, Schafers M, Reuter S. Strategies for non-invasive molecular imaging of acute allograft rejection by gamma scintigraphy and positron emission tomography. Curr Radiopharm. 2011; 4:10-23.
(7) Reuter S, Schnockel U, Edemir B et al. Potential of noninvasive serial assessment of acute renal allograft rejection by 18F-FDG PET to monitor treatment efficiency. J Nucl Med. 2010; 51:1644-1652.
(8) Reuter S, Schnockel U, Schroter R et al. Non-invasive imaging of acute renal allograft rejection in rats using small animal F-FDG-PET. PLoS One. 2009; 4:e5296.
(9) Nickeleit V, Andreoni K. Inflammatory cells in renal allografts. Front Biosci. 2008; 13:6202-6213.
(10) Cornell L D, Smith R N, Colvin R B. Kidney transplantation: mechanisms of rejection and acceptance. Annu Rev Pathol. 2008; 3:189-220.
(11) Ingulli E. Mechanism of cellular rejection in transplantation. Pediatr Nephrol. 2010; 25:61-74.
(12) Velic A, Gabriels G, Hirsch J R et al. Acute rejection after rat renal transplantation leads to downregulation of NA+ and water channels in the collecting duct. Am J Transplant. 2005; 5:1276-1285.
(13) Velic A, Hirsch J R, Bartel J et al. Renal transplantation modulates expression and function of receptors and transporters of rat proximal tubules. J Am Soc Nephrol. 2004; 15:967-977.
(14) Schnockel U, Reuter S, Stegger L et al. Dynamic 18F-fluoride small animal PET to noninvasively assess renal function in rats. Eur J Nucl Med Mol Imaging. 2008; 35:2267-2274.
(15) Schafers K P, Reader A J, Kriens M, Knoess C, Schober O, Schafers M. Performance evaluation of the 32-module quadHIDAC small-animal PET scanner. J Nucl Med. 2005; 46:996-1004.
(16) Sis B, Sarioglu S, Celik A et al. Renal medullary changes in renal allograft recipients with raised serum creatinine. J Clin Pathol. 2006; 59:377-381.
(17) Feige J N, Sage D, Wahli W, Desvergne B, Gelman L. PixFRET, an ImageJ plug-in for FRET calculation that can accommodate variations in spectral bleed-throughs. Microsc Res Tech. 2005; 68:51-58.
(18) Di Marco G S, Reuter S, Hillebrand U et al. The soluble VEGF receptor sFlt1 contributes to endothelial dysfunction in CKD. J Am Soc Nephrol. 2009; 20:2235-2245.
(19) Sarwal M, Chua M S, Kambham N et al. Molecular heterogeneity in acute renal allograft rejection identified by DNA microarray profiling. N Engl J Med. 2003; 349:125-138.
(20) Edemir B, Reuter S, Borgulya R et al. Acute rejection modulates gene expression in the collecting duct. J Am Soc Nephrol. 2008; 19:538-546.
(21) Solez K, Colvin R B, Racusen L C et al. Banff 07 classification of renal allograft pathology: updates and future directions. Am J Transplant. 2008; 8:753-760.
(22) Sis B, Mengel M, Haas M et al. Banff '09 meeting report: antibody mediated graft deterioration and implementation of Banff working groups. Am J Transplant. 2010; 10:464-471.
(23) Reuter S, Rahbar K, Busch V et al. Acute renal failure due to primary bilateral renal large B-cell lymphoma: diagnostics and follow-up by FDG-PET/CT. Clin Nucl Med. 2009; 34:722-724.
(24) Toso C, Zaidi H, Morel P et al. Assessment of 18F-FDG-leukocyte imaging to monitor rejection after pancreatic islet transplantation. Transplant Proc. 2006; 38:3033-3034.
(25) Fu Y, Maianu L, Melbert B R, Garvey W T. Facilitative glucose transporter gene expression in human lymphocytes, monocytes, and macrophages: a role for GLUT isoforms 1, 3, and 5 in the immune response and foam cell formation. Blood Cells Mol Dis. 2004; 32:182-190.
(26) Maratou E, Dimitriadis G, Kollias A et al. Glucose transporter expression on the plasma membrane of resting and activated white blood cells. Eur J Clin Invest. 2007; 37:282-290.
(27) Botti C, Negri D R, Seregni E et al. Comparison of three different methods for radiolabelling human activated T lymphocytes. Eur J Nucl Med. 1997; 24:497-504.
(28) Forstrom L A, Dunn W L, Mullan B P, Hung J C, Lowe V J, Thorson L M. Biodistribution and dosimetry of [(18)F]fluorodeoxyglucose labelled leukocytes in normal human subjects. Nucl Med Commun. 2002; 23:721-725.
(29) Pellegrino D, Bonab A A, Dragotakes S C, Pitman J T, Mariani G, Carter E A. Inflammation and infection: imaging properties of 18F-FDG-labeled white blood cells versus 18F-FDG. J Nucl Med. 2005; 46:1522-1530.
(30) Wang R, Zhang J, Guo Z et al. In-vivo PET imaging of implanted human retinal pigment epithelium cells in a Parkinson's disease rat model. Nucl Med Commun. 2008; 29:455-461.
(31) Hay R V, Skinner R S, Newman O C et al. Scintigraphy of acute inflammatory lesions in rats with radiolabelled recombinant human interleukin-8. Nucl Med Commun. 1997; 18:367-378.
(32) Dumarey N, Egrise D, Blocklet D et al. Imaging infection with 18F-FDG-labeled leukocyte PET/CT: initial experience in 21 patients. J Nucl Med. 2006; 47:625-632.
(33) Dumarey N. Imaging with FDG labeled leukocytes: is it clinically useful? Q J Nucl Med Mol Imaging. 2009; 53:89-94.
(34) Peters A M, Danpure H J, Osman S et al. Clinical experience with 99mTc-hexamethylpropylene-amineoxime for labelling leucocytes and imaging inflammation. Lancet. 1986; 2:946-949.
(35) Papos M, Nagy F, Lang J, Csernay L. Leukocyte scintigraphy to assess disease activity in inflammatory bowel disease. J Nucl Med. 1996; 37:400.
(36) Watson C J, Wraight E P, Neale G, Jamieson N V, Friend P J, Calne R. Radionuclide studies in intestinal transplantation. Diagnosis of rejection and assessment of permeability. Transplantation. 1996; 61:155-157.
(37) Lopes de Souza S A, Barbosa da Fonseca L M, Torres G R et al. Diagnosis of renal allograft rejection and acute tubular necrosis by 99mTc-mononuclear leukocyte imaging. Transplant Proc. 2004; 36:2997-3001.

### TABLES

TABLE 1
Sequences of primers used for RT-PCR

| Gene | Primer-Sense | Primer-Antisense |
|---|---|---|
| Gapdh | CAT CAACGACCCCTTCA | ACTCCACGACATACTCAG |
| | TTGAC | CACC |
| | (SEQ ID No. 5) | (SEQ ID No. 6) |
| CD3d | TTCAAGATAGAAGTGGTT | CACCTCCTTCGCCAGCTC |
| | GAATATG | C |
| | (SEQ ID No. 7) | (SEQ ID No. 8) |
| CD8b | GCTTGACATGTGGCCTCA | CGTGGGCGCGGATCATTT |
| | TTAC | GTG |
| | (SEQ ID No. 9) | (SEQ ID No. 10) |

TABLE 2
Accumulation of ¹⁸F-FDG labeled T-lymphocytes
(autoradiographic analysis)

| | aTX | sTX | IRI | CSA |
|---|---|---|---|---|
| Cortex | 3.98 * | 1.38 | 1.12 | 0.91 |
| Medulla | 3.76 * | 1.53 | 1.26 | 0.85 |

Accumulation of radiolabeled T-cells in the renal medulla and cortex was assessed by autoradiography, expressed as radioactivity ratio of the respective tissue sample and native controls.
aTX: allogeneic transplant, sTX: syngeneic transplant, IRI: ischemia/reperfusion injury, CSA: Cyclosporin A toxicity. (n = 5)
(* P < 0.001).

TABLE 3
Real Time PCR-based characterization of T-lymphocyte infiltration
in different groups of kidneys on day 4 after surgery.

| Cell type | mRNA | aTX | sTX | IRI | CSA |
|---|---|---|---|---|---|
| T-cells | CD3 | 100.6 ± 12.7* | 2.4 ± 0.3 | 3.2 ± 0.3 | 1.5 ± 0.2 |
| Cytotoxic | CD8b | 115.8 ± 12.6* | 1.4 ± 0.2 | 2.3 ± 0.2 | 1.6 ± 0.5 |
| T-cells | | | | | |

*Significantly increased relative to control (P < 0.0001)
Data are mean values (compared with control values, n = 4-6) ± SEM.

1. A method of detecting acute renal allograft rejection in a subject, the method comprising:
(a) administering to the subject a probe capable of specifically binding to T lymphocytes, and wherein the probe is detectable by ultrasound;
(b) exposing an allotransplanted kidney of the subject to ultrasound; and
(c) detecting a level of T lymphocytes in the kidney;
wherein an elevated level of T lymphocytes in the kidney indicates acute renal allograft rejection.

2. The method of claim 1, wherein the probe has a binding molecule coupled thereto, wherein the binding molecule is specific for T lymphocytes.

3. The method of claim 1, wherein the probe is a nanoparticle or a microvesicle.

4. The method of claim 2, wherein the binding molecule is specific for a molecule or a moiety present on T lymphocytes.

5. The method of claim 4, wherein the molecule present on T lymphocytes is one of CD3, CD4, CD8, CD154, CTLA-4 and CD62L.

6. The method of claim 4, wherein the binding molecule is an immunoglobulin or a proteinaceous binding molecule with immunoglobulin-like functions specific for the molecule or the moiety present on T lymphocytes.

7. The method of claim 1, wherein detecting the level of T lymphocytes in the kidney is carried out by means of an imaging technique.

8. The method of claim 1, wherein detecting the level of T lymphocytes in the kidney comprises comparing the detected level of T lymphocytes to a threshold value.

9. The method of claim 8, wherein the threshold value is based on the level of T lymphocytes in a homologous kidney of the subject.

10. The method of claim 8, wherein the threshold value is based on the level of T lymphocytes in a homologous kidney of a control subject.

11. The method of claim 1, wherein detecting the level of T lymphocytes in the kidney is carried out by a real-time measurement.

12. The method of claim 1, wherein exposing the allotransplanted kidney to ultrasound is carried out using a high intensity focused ultrasound technique.

13. The method of claim 1, wherein the subject is a mammal.

14. A probe for detection of acute renal allograft rejection in a subject, wherein the probe is capable of specifically binding to T lymphocytes and is detectable by ultrasound, and wherein the detection comprises:
(a) administration of the probe to the subject;
(b) exposure of an allotransplanted kidney of the subject to ultrasound; and
(c) detection of a level of T lymphocytes in the kidney;
wherein an elevated level of T lymphocytes in the kidney indicates an increased risk of renal allograft rejection.

15. The probe of claim 14, wherein the probe has a binding molecule coupled thereto, wherein the binding molecule is specific for T lymphocytes.

16. The probe of claim 14, wherein the probe is a nanoparticle or a microvesicle.

17. The probe of claim 15, wherein the binding molecule is specific for a molecule or a moiety present on T lymphocytes.

18. The probe of claim 14, wherein the detected T lymphocytes are CD3+T lymphocytes.

19-22. (canceled)

23. A method of treating renal allograft rejection in a subject having received a renal allograft, the method comprising:
(a) administering to the subject a probe capable of specifically binding to T lymphocytes wherein the probe is detectable by ultrasound;
(b) exposing an allotransplanted kidney of the subject to ultrasound;
(c) detecting the level of T lymphocytes in the kidney; and
(d) adapting or starting an immunosuppressive therapy to the subject if an elevated level of T lymphocytes in the kidney is detected, and
not adapting or starting an immunosuppressive therapy to the subject if no elevated level or a decreased level of T lymphocytes in the kidney is detected.

24. The method of claim 23, wherein starting an immunosuppressive therapy comprises administering an immunosuppressive agent to the subject.

25-36. (canceled)


SEQUENCE LISTING (10 sequences)

SEQ ID NO: 1 - protein, 10 aa, artificial
  T7 epitope
ASMTGGQQMG

SEQ ID NO: 2 - protein, 11 aa, artificial
  HSV epitope of glycoprotein D
QPELAPEDPED

SEQ ID NO: 3 - protein, 9 aa, artificial
  HA epitope
YPYDVPDYA

SEQ ID NO: 4 - protein, 10 aa, artificial
  myc epitope
EQKLISEEDL

SEQ ID NO: 5 - DNA, 22 bp, artificial
  GAPDH primer
catcaacgaccccttcattgac

SEQ ID NO: 6 - DNA, 22 bp, artificial
  GAPDH primer
actccacgacatactcagcacc

SEQ ID NO: 7 - DNA, 25 bp, artificial
  CD3d primer
ttcaagatagaagtggttgaatatg

SEQ ID NO: 8 - DNA, 19 bp, artificial
  CD3d primer
cacctccttcgccagctcc

SEQ ID NO: 9 - DNA, 22 bp, artificial
  CD8b primer
gcttgacatgtggcctcattac

SEQ ID NO: 10 - DNA, 21 bp, artificial
  CD8b primer
cgtgggcgcggatcatttgtg